(19) United States
(12) Patent Application Publication
(10) Pub. No.: US 2023/0355812 A1
(43) Pub. Date: Nov. 9, 2023

(54) STABILIZED HYDROPHOBIC NANOPARTICLES FOR ULTRASOUND IMAGING

(71) Applicant: Oregon Health & Science University, Portland, OR (US)

(72) Inventors: Adem YILDIRIM, Portland, OR (US); Jose Luis Montoya MIRA, Portland, OR (US); Sadik ESENER, Portland, OR (US); Stuart IBSEN, Portland, OR (US); Jared M. FISCHER, Portland, OR (US); Sinan SABUNCU, Portland, OR (US); Elise MANALO-HALL, Portland, OR (US)

(21) Appl. No.: 18/044,444

(22) PCT Filed: Sep. 8, 2021
(86) PCT No.: PCT/US21/49518

§ 371 Date: Mar. 8, 2023

Related U.S. Application Data
(60) Provisional application No. 63/075,789, filed on Sep. 8, 2020.
(60) Provisional application No. 63/230,623, filed on Aug. 6, 2021.

(51) Int. Cl. A61K 49/22; A61K 9/51; A61K 41/00; A61K 9/50; A61B 8/08

(52) U.S. Cl. CPC A61K 49/225; A61K 9/5192; A61K 41/0033; A61K 9/5052; A61K 9/5031; A61K 9/5115; A61B 8/481

(57) ABSTRACT

Disclosed are nanoparticle-based ultrasound contrast agents that comprise a sub-100 nanometer nanoparticle core, a hydrophobic layer, and a stabilization layer, and methods of producing such ultrasound contrast agents. The stabilization layer comprises molecules that are spaced apart on the stabilization layer to provide bubble nucleation sites that initiate cavitation of the bubbles in response to acoustic intensity delivered by ultrasound equipment.

### RELATED APPLICATIONS

This application claims priority to U.S. Provisional Application No. 63/075,789, filed Sep. 8, 2020, and titled AMPHIPHILIC STABILIZED HYDROPHOBIC NANOPARTICLES FOR ULTRASOUND IMAGING, and U.S. Provisional Application No. 63/230,623, filed Aug. 6, 2021, and titled BIODEGRADABLE GAS STABILIZING NANOPARTICLES FOR TUMOR ABLATION, each of which is incorporated herein by reference in its entirety.

### TECHNICAL FIELD

This disclosure relates generally to ultrasound contrast agents, and more particularly, to nanoparticle ultrasound contrast agents having stabilizing molecule coatings.

### BACKGROUND INFORMATION

Ultrasound contrast agents (UCAs) can enhance the ultrasound signal and enable molecular imaging of the disease-related biomarkers. UCAs include microbubble ultrasound contrast agents, nanobubbles, nanodroplets, echogenic liposomes, and nanoparticle-based contrast agents. Some UCAs, such as stabilized perfluorocarbon microbubbles or nanodroplets, are larger than 200 nanometers (nm) and are unstable to extravasate into the targeted solid tumor effectively. Thus, their accumulation in solid tumors is low and usually insufficient for ultrasound detection and molecular imaging.

UCAs based on hydrophobic nanoparticles have been developed in some recent studies. Hydrophobic mesoporous silica nanoparticles stabilized by F127 copolymer or phospholipids can demonstrate acoustic cavitation generation by using high intensity focused ultrasound (HIFU). Others have prepared amphiphilic stabilized hydrophobic mesoporous silica nanoparticles to increase the efficiency of sonodynamic therapy. In another study, gold nanorods coated with amphiphilic peptides were shown to generate ultrasound contrast when five mg/mL were used. Another study group engineered the surfaces of mesoporous silicon nanoparticles to develop ultrasound contrast agents. While the particles can generate ultrasound contrast at low particle concentrations, the synthesis of the particles require electrochemical etching of silicon wafers, use sonication to form the particles, and cannot be applied other types of particles. Other ultrasound contrast agent-based nanoparticles use polymer nanocups or gold nanocones as nucleation sites to generate acoustic cavitation and applied them for drug delivery; however, their surfaces are not yet suitable for further functionalization. There are methods to prepare gas stabilizing nanoparticles (GSNs) with hydrophilic (PEGylated) and functionalizable surfaces by coating hydrophobically modified mesoporous silica nanoparticles (MSNs) with amphiphilic polymers or phospholipids. However, the GSNs prepared in these studies required high acoustic pressures (peak negative pressure, PNP>5 MPa, corresponding mechanical index, MI, of ˜5) to nucleate the echogenic bubbles. Thus, a high intensity focused ultrasound (HIFU) transducer was necessary to provide the required high acoustic pressures, which not only complicates the instrumentation for ultrasound imaging of these GSNs but also limits the imaging volume to the small focal zone of HIFU transducers.

A typical method for coating hydrophobic nanoparticles with amphiphilic molecules (amphiphilic block copolymers, surfactants, phospholipids, etc.) is the phase transfer from the organic phase (containing nanoparticles and may also contain amphiphilic molecules) to the water phase (may contain amphiphilic molecules). For phase transfer, sonication or stirring can be used. Alternatively, the organic phase can be evaporated by heating, under vacuum, or by the gas flow. Another common way is to mix nanoparticles and amphiphiles in an organic solvent first and then evaporate the solvent to obtain a thin film of nanoparticles and amphiphilic molecules. Then the film is suspended in an aqueous solution by sonication or stirring. Another method is to add dry particles onto an aqueous solution containing amphiphilic molecules. For the phase transfer, particles are vigorously stirred and/or sonicated for long times. The extensive sonication or heating can destabilize the air-pockets nucleated on the nanoparticles; thus, reduce their acoustic activity. In fact, it was observed that the protocols that include bath sonication longer than approximately 1 minute (min) yield contrast agents with weak acoustic activity. Such contrast agents can be only activated at high pressures by using a high intensity focused ultrasound transducer.

### SUMMARY OF THE DISCLOSURE

Disclosed are stabilized hydrophobic nanoparticles for ultrasound imaging contrast agents that are capable of being imaged by ultrasound equipment delivering acoustic intensity at a mechanical index of about 1.9 or less. The stabilized hydrophobic nanoparticles comprise a sub-100 nanometer nanoparticle core having an outer surface, a silane layer coating the outer surface of the sub-100 nanometer nanoparticle core, and a stabilizing layer comprising stabilizing molecules. Each stabilizing molecule has a binding portion bound to the silane layer and a non-binding portion free from the silane layer, the binding portions of different ones of stabilizing molecules being spaced apart on the silane layer by distances configured to provide bubble nucleation sites that, in response to the ultrasound equipment delivering the acoustic intensity at the mechanical index of about 1.9 or less, initiate cavitation of echogenic micron-sized bubbles. The sub-100 nanometer nanoparticle core may comprise silicon, gold, silver, iron oxide, titanium dioxide, carbon, organosilica, a polymer, platinum, metal-organic framework, hydrogel, polydopamine, cellulose, or mesoporous silica. The sub-100 nanometer nanoparticle may be about 50 nanometers. The silane layer coating the outer surface of the nanoparticle core may comprise chlorosilanes, methoxysilanes, ethoxysilanes, disilazanes, or hexamethyldisilazane. The stabilizing molecules may be amphiphilic molecule chains, such as amphiphilic block polymers e.g., poloxamers, and phospholipids. The stabilizing molecules may be a protein, such as albumin. Methods of using such stabilized hydrophobic nanoparticles are also described.

Also disclosed are methods of preparing stabilized hydrophobic nanoparticle-based ultrasound imaging contrast agents that are capable of being imaged by ultrasound equipment delivering acoustic intensity at a mechanical index of about 1.9 or less. The methods can comprise adding an amount of amphiphilic molecule to a dried film of hydrophobic nanoparticles, where the amount of amphiphilic molecule has a quantity of amphiphilic molecules configured to facilitate spaced-apart surface binding of the amphiphilic molecules on the hydrophobic nanoparticles by distances that provide bubble nucleation sites that, in response to the ultrasound equipment delivering the acoustic intensity at the mechanical index of about 1.9 or less, initiate cavitation of echogenic micron-sized bubbles. Further, the methods include sonicating the dried film of hydrophobic nanoparticles and the amphiphilic molecule concentration together for about five seconds to coat the hydrophobic nanoparticles with the amphiphilic molecules and thereby form a colloidal, dispersed suspension of amphiphilic stabilized hydrophobic nanoparticles. Instead of amphiphilic molecules, a dried film of hydrophobic molecules may be sonicated in presence of water then mixed with a protein solution. The methods may further comprise synthesizing a sub-100 nanometer nanoparticle core having an outer surface, modifying the outer surface of the sub-100 nanometer nanoparticle core with silane monomers, and drying the sub-100 nanometer nanoparticle core having the silane monomers on the outer surface of the sub-100 nanoparticle core to form the dried film of hydrophobic nanoparticles. In these methods, the stabilizing molecule can be an amphiphilic molecule such as poloxamer 407 or a protein molecule such as serum albumin.

Also disclosed are methods of using the stabilized hydrophobic nanoparticles described herein in ultrasound imaging and therapy. A method of using the hydrophobic nanoparticles as an ultrasound imaging contrast agent can comprise administering the hydrophobic nanoparticle to a subject at a concentration in a range of about 1 μg/mL to about 5 mg/mL. A method of using the hydrophobic nanoparticles in HIFU ablation therapy can comprise steps of a) delivering the hydrophobic nanoparticle to a target tissue at a concentration of about 0.05 mg/mL to about 10 mg/mL and b) insonating the target tissue with HIFU to reduce a volume of the target tissue.

Additional aspects and advantages will be apparent from the following detailed description of preferred embodiments, which proceeds with reference to the accompanying drawings.

### BRIEF DESCRIPTION OF THE DRAWINGS

FIG. 1 depicts steps of an ultrasound contrast agent preparation process using hydrophobically modified nanoparticles according to an embodiment.

FIGS. 2A-2B show a schematic representation of (FIG. 2A) hydrophobic surface modification of nanoparticles and their stabilization by amphiphilic molecules, (FIG. 2B) microbubble generation by the nanoparticles under reduced acoustic pressures.

FIGS. 3A-3F show representative transmission electron microscopy (TEM) images of nanoparticles that may be used to develop ultrasound contrast agents in accordance with the present disclosure: (FIG. 3A) 100-150 nm solid silica nanoparticles, (FIG. 3B) ˜50 nm mesoporous silica nanoparticles, (FIG. 3C) ˜100 nm mesoporous silica nanoparticles, (FIG. 3D) ˜100 nm large pore dendritic silica nanoparticles, (FIG. 3E) 100-150 nm silica nanocups, and (FIG. 3F) Janus mesoporous silica nanorod coated iron oxide nanoparticles.

FIGS. 4A-4C show photographs of (FIG. 4A) hydrophobically modified mesoporous silica nanoparticles floating on water, (FIG. 4B) a water droplet sitting on the thin film formed by drying hydrophobic nanoparticles in a glass vial, and (FIG. 4C) colloidal suspension of F127 stabilized hydrophobic nanoparticles in PBS.

FIG. 5 shows Raman spectra of mesoporous silica nanoparticles (MSN), hydrophobic mesoporous silica nanoparticles (hMSN), poloxamer 407 (F127)-coated hMSN (F127-hMSN), and poloxamer 407 samples.

FIG. 6 is a schematic representation of the proposed mechanism for highly-echogenic gas stabilizing nanoparticle synthesis. At low F127 concentrations, the particles are poorly covered with the amphiphilic polymer, which results in aggregates in PBS (panel i). High F127 concentrations yield full coverage of particle surfaces with the amphiphilic polymer, thus prevents nanobubble formation (panel iii). When optimal amounts of F127 are used, it is possible to prepare stable colloidal solutions of hMSN with stabilized surface nanobubbles (panel ii).

FIGS. 7A-7D show graphical representations of: (FIG. 7A) dynamic light scattering (DLS) analysis of F127-hMSN samples prepared in PBS (10 mM, pH 7.4) using different amounts of F127; (FIG. 7B) calculated intensities from acquired videos during ultrasound imaging (2.5 MHz, MI=1.4) of F127-hMSN samples dispersed in PBS at a particle concentration of 100 μg/mL, where MSN and F127 are control samples of unmodified mesoporous silica nanoparticles (dispersed in water, 100 μg/mL) and 0.05 mg/mL F127 in PBS in the absence of any particles, respectively; (FIG. 7C) effect of particle concentration on the intensity generated (at 2.5 MHz, and MI=1.4) by F127-hMSN sample prepared using 0.5 mg/mL of F127; and (FIG. 7D) signal intensity plot of the F127-hMSN sample (stabilized using 0.5 mg/mL F127) in PBS before and after rapidly increasing the MI to 1.4 from 0.2. Insets show the representative B-mode ultrasound images, and red squares in the images highlight the region of interest. Error bars=Standard error of the mean, studies were run as technical triplicates. According to Student's t-test, n.s.=non-significant, *p<0.05, **p<0.01, and ***p<0.001. Student's t-test was performed against control (PBS).

FIG. 8 is a graphical representation of dynamic light scattering (DLS) analysis of F127-hMSN in PBS (10 mM, pH 7.4) at different particle concentrations.

FIG. 9 is a graph of mechanical index versus the intensity generated (at 2.5 MHz) by F127-hMSN (100 μg/mL) in PBS prepared using 0.5 mg/mL of F127. Average intensities calculated from the acquired movies of samples. Error bars=Standard error of the mean, studies were run in triplicate.

FIGS. 10A and 10B show graphical representations of the mechanical index (MI) versus the intensity generated, at the particle concentration of 100 μg/mL, (FIG. 10A) and particle concentration, at the MI of 1.4, (FIG. 10B) on the contrast enhancement by 50 nm hydrophobic mesoporous silica nanoparticles stabilized using 0.5 mg/mL F127. Average intensities calculated from the acquired movies of samples. Error bars=Standard error of the mean, studies were run in triplicate.

FIGS. 11A-11C show graphical representations of (FIG. 11A) signal intensity plot of F127-hMSN in PBS (100 μg/mL) under continuous ultrasound imaging (2.5 MHz) for ˜23 min, (FIG. 11B) the first 1 min of the data presented in panel a showing the increase in the intensity of the ultrasound signal after gradually increasing the MI, where the arrows indicate the time points where MI was increased, and (FIG. 11C) signal intensity plot of PBS in the absence of F127-hMSN.

FIGS. 12A-12C show graphs of intensity generated by F127-hMSN samples after overnight incubation in 50% serum at 37° C. and in tissue-mimicking agarose gel phantoms. Calculated intensities from acquired videos of F127-hMSN samples (100 μg/mL) dispersed in PBS or 50% serum (FIG. 12A) during ultrasound imaging and (FIG. 12B) under HIFU insonation and (FIG. 12C) F127-hMSN samples (100 μg/mL) dispersed in PBS or in 1% (w/w) agarose gel phantoms are shown. Insets show the representative B-mode ultrasound images and red squares or circles in the images highlight the region of interest. Average intensities calculated from the acquired movies of samples. Error bars=Standard error of the mean, studies were run as technical triplicates. According to Student's t-test, n.s.=non-significant and *p<0.05.

FIG. 13 is an image showing F127 coated hydrophobic mesoporous silica nanoparticles in simulated body fluid after 1 week incubation at 37° C.

FIGS. 14A and 14B show (FIG. 14A) a schematic representation of the experimental set-up used for high intensity focused ultrasound (HIFU) insonation and (FIG. 14B) a graphical representation of calculated intensities from acquired videos during ultrasound imaging (5 MHz, MI=0.26) of F127-hMSN (100 μg/mL) samples prepared in PBS using different amounts of F127 under HIFU insonation (40 W). MSN and F127 were control samples of unmodified mesoporous silica nanoparticles (dispersed in water, 100 μg/mL) and 0.05 mg/mL F127 in PBS in the absence of any particles, respectively. Insets show the representative B-mode ultrasound images, and red circles in the images highlight the region of interest. Error bars=Standard error of the mean, studies were run as technical triplicates. According to Student's t-test, n.s.=non-significant and **p<0.01. Student's t-test was performed against control (PBS).

FIG. 15 is a graph of the calculated intensities from acquired videos during ultrasound imaging (5 MHz, MI=0.26) of F127-hMSN (100 μg/mL) samples under HIFU insonation at different electrical power inputs. Error bars=Standard error of the mean, studies were run in triplicate.

FIG. 16 is a signal intensity plot of the F127-hMSN sample (100 μg/mL) in PBS under HIFU insonation (40 W).

FIG. 17 is a signal intensity plot of the F127-hMSN sample (100 μg/mL) in PBS under HIFU insonation (50 W) for 30 min (three 10 min sonications). Green and red arrows shows the points where sonications were started and stopped, respectively.

FIGS. 18A and 18B show graphs representing calculated intensities from acquired videos during ultrasound imaging (2.5 MHz, MI=1.4) of F127-hMSN dispersed in PBS (100 μg/mL) before and after 5 min of bath sonication (FIG. 18A) and calculated intensities from acquired videos during ultrasound imaging (5 MHz, MI=0.26) of F127-hMSN (100 μg/mL) samples under HIFU insonation (40 W) before and after 5 min of bath sonication (FIG. 18B). Error bars=Standard error of the mean, studies were run in triplicate. PBS is the control sample without any nanoparticles.

FIG. 19 is a graph of additional bath sonication time versus intensity generated by 50 nm hydrophobic mesoporous silica nanoparticles suspensions (stabilized using 0.5 mg/mL F127). Samples (100 μg/mL) were imaged at 2.5 MHz and MI of 1.4. Average intensities calculated from the acquired movies of samples. Error bars=Standard error of the mean, studies were run in triplicate.

FIGS. 20A-20D show graphs of ethidium homodimer (EH) fluorescence versus forward scatter-area (FCS-A) plots of 4T1 cells in the presence or absence of F127-hMSN (100 μg/mL) and with or without ultrasound imaging for 2 min (at 2.5 MHz, MI=1.4) (FIG. 20A), counts (%) for each region (dead cells, debris, viable cells) highlighted in FIG. 20A (FIG. 20B), ethidium homodimer (EH) fluorescence versus forward scatter-area (FCS-A) plots of 4T1 cells in the presence or absence of F127-hMSN (100 μg/mL) after HIFU insonation (FIG. 20C), counts (%) for each region (dead cells, debris, viable cells) highlighted in FIG. 20C. Error bars=Standard error of the mean, studies were run as technical triplicates. According to Student's t-test, n.s.=non-significant, *p<0.05, **p<0.01, and ***p<0.001. Unless otherwise specified Student's t-test was performed against control (no ultrasound insonation and in the absence of F127-hMSN).

FIG. 21 is a graphical representation of the results from a MTS assay of 4T1 cells, which were incubated with F127-hMSN at different particle concentrations. Error bars=Standard error of the mean, 5 wells were used to calculate each data point.

FIG. 22 is a graph of the temperature increase in 4T1 cell suspensions in the presence or absence of F127-hMSN (100 μg/mL) after HIFU insonation for 2 min at different pulse repetition frequencies (50 W). Error bars=Standard error of the mean, studies were run as technical triplicates. According to Student's t-test, n.s.=non-significant.

FIGS. 23A-23C show results of investigating the stability of F127-hMSN particles during storage. FIG. 23A is a graph of calculated intensities from acquired videos during ultrasound imaging (2.5 MHz, MI=1.4) of the F127-hMSN sample (100 μg/mL), which was stored at room conditions in PBS for 1 month at a particle concentration of 1 mg/mL. Inset shows the representative B-mode ultrasound images, and the red square in the image highlights the region of interest. Error bars=Standard error of the mean, studies were run as technical triplicates. FIG. 23B is a graphical representation of DLS analysis of F127-hMSN before and after storing in PBS for 1 month. The inset shows the TEM image of the stored sample. FIG. 23C is an image of F127-hMSN in simulated body fluid after 1 week incubation at 37° C. at a particle concentration of 50 μg/mL.

FIG. 24 is a TEM image of F127-hMSN (50 μg/mL) incubated in simulated body fluid at 37° C. after 4 weeks.

FIG. 25 depicts steps of the ultrasound contrast agent preparation process using hydrophobically modified nanoparticles according to an embodiment.

FIGS. 26A and 26B are a schematic representation of hydrophobic surface modification of mesoporous silica nanoparticles (MSNs) and their stabilization by proteins in buffer solutions (FIG. 26A), and microbubble generation by the nanoparticles under reduced acoustic pressures (FIG. 26B).

FIG. 27 shows representative ultrasound images showing contrast generation by bovine serum albumin (BSA) coated hydrophobic MSNs (BSA-hMSN). (Left panel) only PBS, no particles. (Right panel) BSA-hMSN in PBS (100 μg/mL). Dotted squares show the regions of interest. The bright lines at the top and bottom of the images are the reflections that originated from the sample holder walls.

FIGS. 28A and 28B show a signal intensity plot of hydrophobic mesoporous silica nanoparticle (100 μg/mL) suspensions in PBS (10 mM, pH 7.4) stabilized using different protein sources (5 mg/mL) under continuous ultrasound imaging (2.5 MHz) for about 2 minutes where mechanical index (MI) was gradually increased from 0.2 to 1.45 in about the first 10 seconds (FIG. 28A), and a graph of average intensities generated by the particles (FIG. 28B). Error bars=Standard error of the mean. Studies were run in triplicate.

FIG. 29 is a graph of average intensities generated by the particles under HIFU insonation using a transducer operating at 1.1 MHz (power: 100 W, pulse duration: 20 us, pulse repetition frequency: 10 Hz). Error bars=Standard error of the mean. Studies were run in triplicate.

FIGS. 30A and 30B show dynamic light scattering results of nanoparticles stabilized (FIG. 30A) using different proteins (5 mg/mL) and (FIG. 30B) using different amounts of BSA.

FIG. 31A shows transmission electron microscopy (TEM) images of BSA-coated hydrophobic MSNs over 4 days. FIG. 31B shows TEM images of F127-coated hydrophobic MSNs at 0 days and at 28 days post incubation in simulated body fluid.

FIG. 32 shows in vivo imaging of mice that received intramuscular injection of protein-coated nanoparticles (left panels), F127 polymer-coated nanoparticles (center panels), or bare MSNs (right panels) over 54 days.

FIG. 33 shows fluorescence images of various organs of mice that received protein-coated nanoparticles (left panel), F127-coated nanoparticles (center panel), or bare MSNs (right panel), 1 week after tail vein injection.

FIG. 34 is a graph of relative tumor size in nude mice with luciferase-expressing HCT-116 colon cancer xenografts over days after treatment with high-intensity focused ultrasound (HIFU) and mouse serum albumin (MSA)-coated hydrophobic MSNs.

FIG. 35 shows luminescence images of mice with A375 xenograft tumors treated with F127-hMSN (lower panels) or without F127-hMSN (upper panels), taken before (left) and 1 day after (right panels) HIFU treatment. Tumors on right ears (dotted circles) were HIFU treated and tumors on left ears were left as untreated controls.

FIG. 36 shows luminescence images of the mice represented in FIG. 34 that were injected with (center and lower rows) or without (upper row) mouse albumin coated hydrophobic MSNs (MSA-hMSN), taken before (left panels) and 1 day after (right panels) HIFU treatment. Tumors enclosed in dotted circles were treated with HIFU. Tumors not enclosed with a dotted circle were not treated with HIFU.

### DETAILED DESCRIPTION OF PREFERRED EMBODIMENTS

Disclosed herein is a nanoparticle-based ultrasound imaging contrast agent that can be imaged by ultrasound equipment delivering acoustic intensities at a mechanical index of 1.9 or less. In a first aspect, ultrasound imaging contrast agents comprise a stabilized hydrophobic nanoparticle that comprises a sub-100 nanometer nanoparticle core, a silane layer coating the outer surface of the nanoparticle core, and a stabilization layer comprising stabilizing molecules on the silane layer on the surface of the nanoparticle core. The stabilizing molecules, individually an amphiphilic molecule chain or a protein, have a binding portion or a hydrophobic portion that are capable of binding to a hydrophobic layer and a non-binding portion or a hydrophilic portion that remains free from a hydrophobic layer. Individual stabilizing molecules are bound to the hydrophobic layer on the outer surface of the nanoparticle core, spaced apart by a distance suitable for providing bubble nucleation sites that initiate cavitation of echogenic micron-sized bubbles in response to acoustic intensities delivered at a mechanical index of about 1.9 or less.

The nanoparticles disclosed herein may be used as an ultrasound imaging contrast agent by administering the nanoparticle to a subject at concentration of about 1 μg/mL to about 5 mg/m L or about 1 μg/mL to about 2 mg/m L or about 2.5 μg/mL to about 100 μg/mL.

Also disclosed herein are methods of preparing stabilized hydrophobic nanoparticles for an ultrasound imaging contrast agent capable of being imaged by ultrasound equipment. In a second aspect, methods of preparing stabilized hydrophobic nanoparticles include treating the outer surface of a sub-100 nanometer nanoparticle core with one or more silane monomers, dispersing the hydrophobic nanoparticle in an organic solvent, removing the organic solvent to provide a dried nanoparticle film, and treating the dried nanoparticle film with a stabilizing molecule solution. The one or more silane monomers are selected from chlorosilanes, methoxysilanes, ethoxysilanes, and disilazanes. The final particle nanoparticle concentration dispersed in the organic solvent ranges from about 1 mg/mL to about 6 mg/mL, or about 2 mg/mL to about 4 mg/mL, or about 1 mg/mL to about 3 mg/mL, or about 3 mg/m L to about 6 mg/m L.

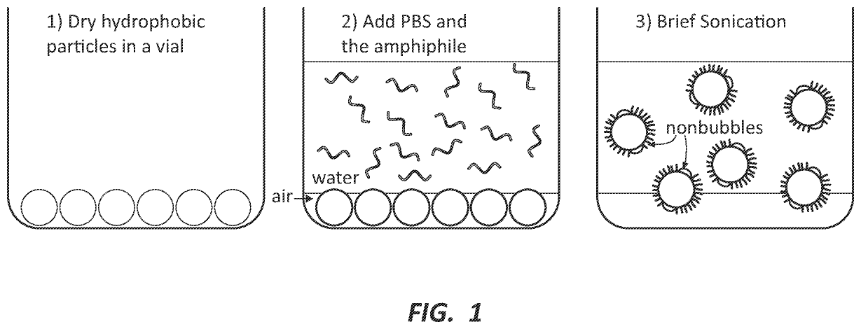

In some embodiments, methods of preparing stabilized hydrophobic nanoparticles include adding amphiphilic molecules to dried hydrophobic nanoparticles then obtaining stable colloidal suspensions after brief bath sonication for approximately 5 seconds (s). No washing step is required since small amounts of biocompatible amphiphilic molecules are used (FIG. 1). This coating method can prepare highly responsive contrast agents that can be imaged using conventional medical ultrasound instruments.

In some embodiments, methods of preparing stabilized hydrophobic nanoparticles include forming a dried nanoparticle film as described above, where treating the dried nanoparticle film can include adding water to the dried nanoparticle film, sonicating the water and the dried nanoparticle film to produce a suspension of hydrophobic nanoparticles, and adding a protein solution to the suspension of the hydrophobic nanoparticles.

Generally, a limitation of nanoparticle-based contrast agents (such as metal nanoparticles or inorganic quantum dots) is their inability to degrade in in vivo conditions. By using biodegradable nanoparticles (such as organosilane nanoparticles or dendritic mesoporous silica nanoparticles), it is possible to prepare an ultrasound contrast agent that degrades in the body in a reasonable time frame (less than about one month, for example, in about 1 week, about 2 weeks, about 3 weeks). It should be noted that the degradation preferably does not occur quickly, for example in less than about 5 hours. Preferably, contrast agents should remain intact until they accumulate in the imaging area, and then they should slowly degrade in the next few days. The contrast agent disclosed herein can be formulated to be biodegradable in accordance with these considerations.

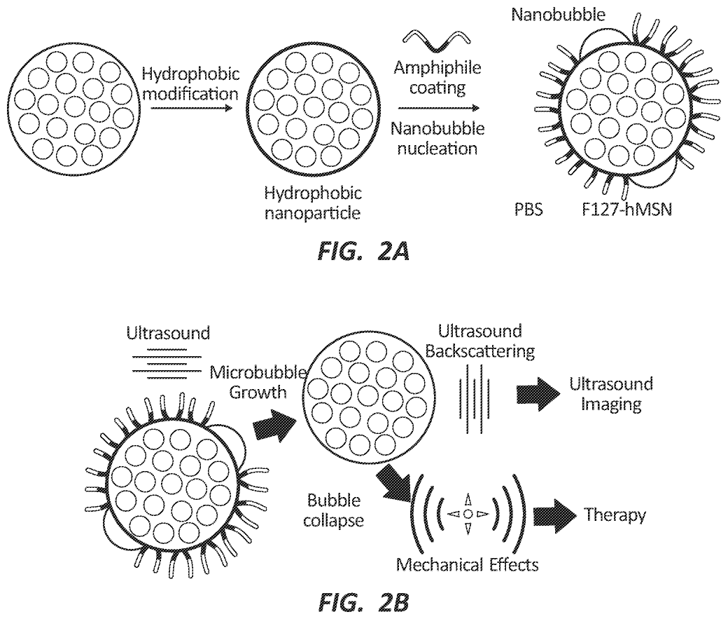

The hydrophobic interfaces of the particles may enable the stabilization of air-pockets at the particle surface, which may, in turn, nucleate acoustic cavitation events under reduced acoustic pressures, where echogenic micron-sized bubbles are generated (FIGS. 2A and 2B). Contrast agents using nanoparticles with sizes as small as 50 nm were prepared, but even smaller contrast agents (approximately 10 nm) can be developed using smaller nanoparticles. In an embodiment, the nanoparticle core is about 50 nanometers. The diameter of the sub-100 nm nanoparticle core may be from about 30 nm to about 90 nm, about 30 nm to about 70 nm, about 40 nm to about 60 nm, about 45 nm to about 55 nm, or about 50 nm.

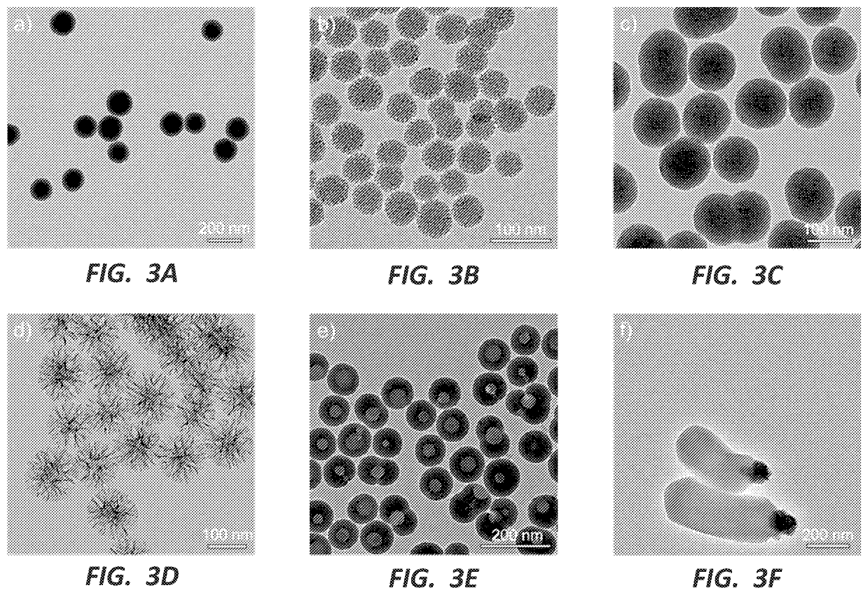

To prepare the contrast agents, firstly, silica nanoparticle cores with different morphologies are synthesized (FIGS. 3A-3F). Instead of silica, the nanoparticle core can comprise other materials. The composition of the nanoparticle core may be based on porous, non-porous nanoparticles, nanoparticles with rough surfaces, or any kind of solid particle (gold, silver, iron oxide, titanium dioxide, carbon, organosilica, polymer, platinum, metal organic frameworks, hydrogels, polydopamine etc.). In addition, particles may have different morphologies (porous, non-porous, core-shell, hollow, etc.) and shapes (spherical, dendritic, rods, plates, cups, etc.). By using degradable nanoparticles (such as dendritic silica, organosilica, polymer nanoparticles, etc.) in in vivo conditions in a reasonable time period (less than one month), it can also be possible to prepare biodegradable ultrasound contrast agents, which is important in terms of clinical applications of these contrast agents.

In an embodiment, the nanoparticle core is mesoporous silica. After synthesizing the nanoparticle core, the surfaces of nanoparticle cores can be hydrophobically modified using reactive silane monomers (such as chlorosilanes, methoxysilanes, ethoxysilanes, mono-alkyl silanes, fluoroalkyl silanes, disilazanes, and other hydrophobic silanes). In an embodiment, the silane layer of the nanoparticles comprises chlorosilanes, methoxysilanes, ethoxysilanes, or disilazanes. The silane layer may comprise hexamethyldisilazane. The hydrophobic particles, which may be nanoparticle cores coated with silanes, are then dispersed in a suitable organic solvent (chloroform, ethanol, acetone etc.), and dried in a vial (glass, plastic, metal vials with different sizes and shapes). As will be appreciated by those of skill in the art with the benefit of the present disclosure, the choice of the organic solvent and drying temperature may be selected so as to obtain uniform thin films. For example, a drying temperature may selected that is sufficiently lower than the boiling point of the solvent used, such that the solvent evaporates relatively slowly, e.g. over 2-4 hours.

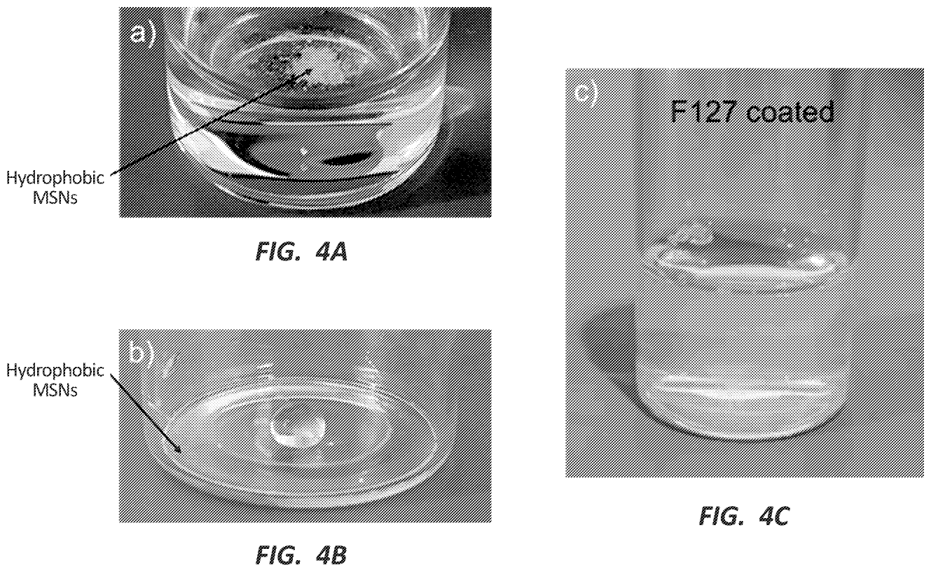

In an exemplary process, hexamethyldisilazene (HMDS) modified 50 nm mesoporous silica nanoparticles could be easily dispersed in ethanol (FIG. 4A), and form uniform thin films when their ethanolic suspensions (1 mL, 4 mg/mL) are dried in 20 mL glass vials at 65° C. (FIG. 4B). The dried films can be further kept at 120° C. to remove any water or other solvents absorbed by the nanoparticles. Alternatively, particles can be kept under vacuum to remove any adsorbed solvents. Finally, buffer solutions containing the amphiphilic molecules (such as amphiphilic block copolymers, surfactants, phospholipids etc.) can be added on the dried particles, and then bath sonicated for ˜5 seconds to form nanoparticle suspensions (FIG. 4C). In an embodiment of the second aspect, the method further comprises synthesizing a sub-100 nanometer nanoparticle having an outer surface, the outer surface of the nanoparticle core hydrophobically modified such as with silane monomers, then the hydrophobically modified nanoparticle dried to form a dried film of hydrophobic nanoparticles. The dried film of hydrophobic nanoparticles are preferably uniform thin films with no cracks or particle clumps visible by eye. The dried hydrophobic nanoparticles may be stored as powders.

To stabilize hydrophobic nanoparticles in aqueous solutions, a Federal Drug Administration (FDA) approved biocompatible amphiphilic polymer, poloxamer 407 (Poly(ethylene glycol)-block-poly(propylene glycol)-block-poly(ethylene glycol), also referred to herein as “F127”), may be used and is shown herein as an example. However, different amphiphilic molecules (such as amphiphilic block copolymers, surfactants, phospholipids, peptide amphiphiles etc) can also be used to stabilize the nanoparticles. Other examples of polymers include poly(lactic-co-glycolic acid), poly(ethylene glycol-block-polylactic acid), poly(ethylene glycol-block-polycaprolactone), and other poloxamers such as poloxamer 188, poloxamer 237, and poloxamer 338. Longer amphiphilic molecules may increase nanoparticle stabilization. Combinations of various polymers may be used. In an embodiment, the stabilizing molecule on hydrophobic nanoparticles of this disclosure can comprise one or more amphiphilic molecule chains selected from poly(glycolic acid) (PGA), Poly(lactic acid) (PLA) and their copolymers, amphiphilic block copolymers such as poloxamers, poly(D, L-lactide-co-glycolide) (PLGA), and phospholipids. Poloxamers include, without limitation, poloxamer 188, poloxamer 237, poloxamer 338, and poloxamer 407. Lactide/glycolide ratios of PLGA can include, without limitation, 50:50, 65:35 and 75:25.

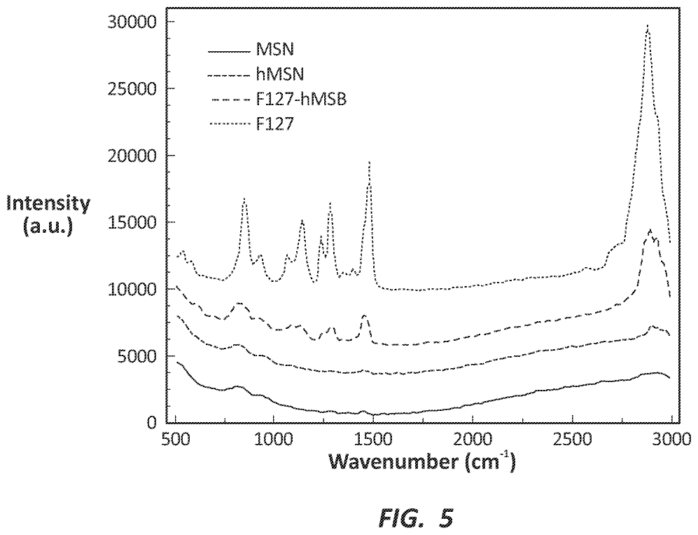

In a continuation of the exemplary process, once the vial was cooled to room temperature, PBS (10 mM, pH 7.4) containing a small amount of F127 (typically 4 mL at a concentration of 0.5 mg/mL) was placed on top of the hydrophobically modified mesoporous silica nanoparticle (hMSN) layer. Finally, the vial was bath sonicated to obtain colloidally stable F127 coated hMSN (hereafter referred to as F127-hMSN) dispersion in PBS (FIG. 4 panel c). It should be noted that the uniformity of the thin films (i. e. no particle aggregates/clumps observable by eye) is important to suspend the hydrophobic particles entirely. Otherwise, some particles that float at the water-air interface after sonication may be observed. Successful HMDS modification of MSNs and capping of hMSNs with F127 were confirmed using Raman spectroscopy (FIG. 5). The broad Raman peak in the C—H stretching mode region between 2800-3000 cm-1 was observed for hMSN, and it was even more intense for F127-hMSN, suggesting the presence of methyl and F127 coatings around the particles. In addition, several peaks between 750-1500 cm-1, corresponding to different modes of C—H, C—C, and C—O bonds, were observed for F127-hMSN, further suggesting the presence of F127 coating around the particles. In an embodiment of the first aspect, the amphiphilic molecules are poloxamers or other amphiphilic block copolymers, or phospholipids. The amphiphilic molecules may be F127.

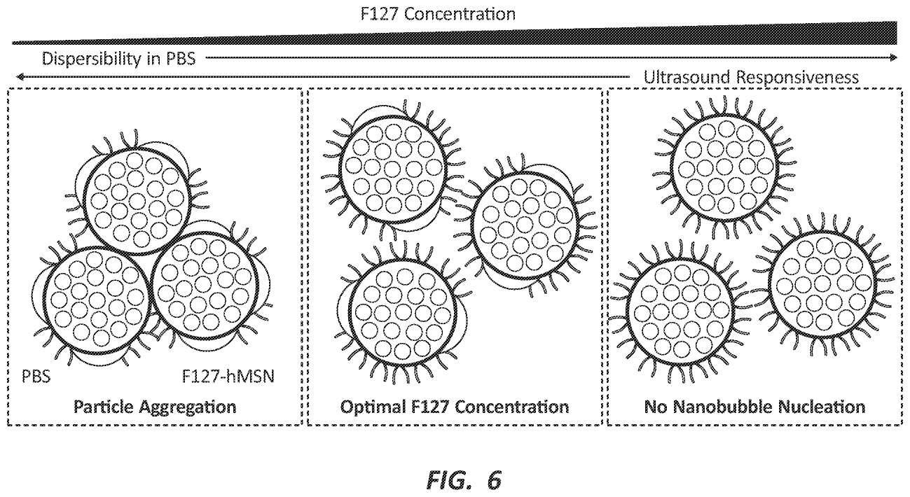

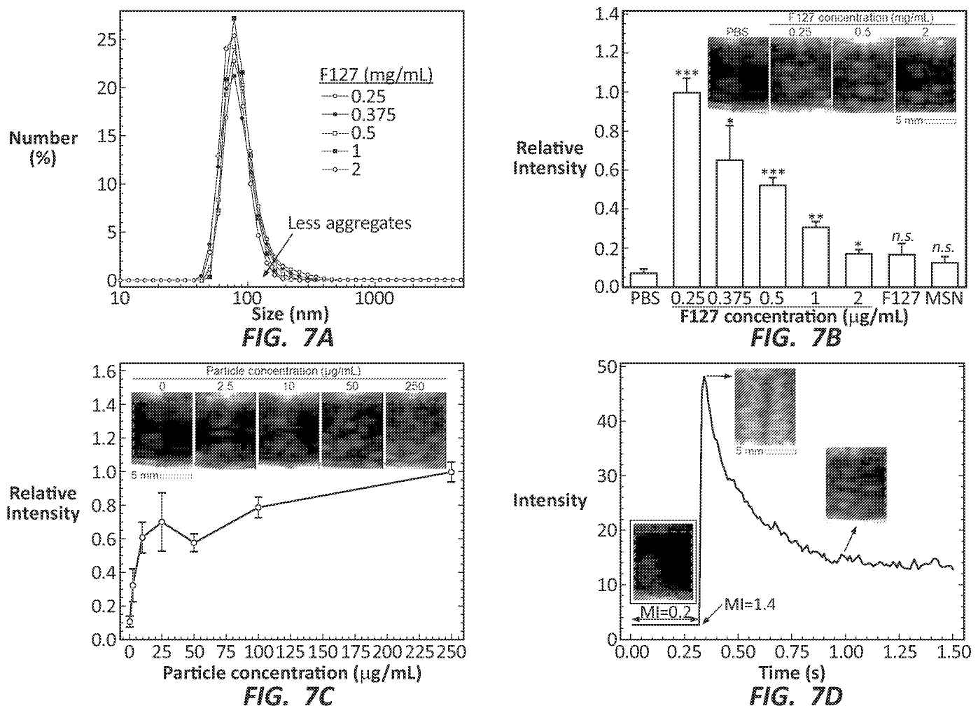

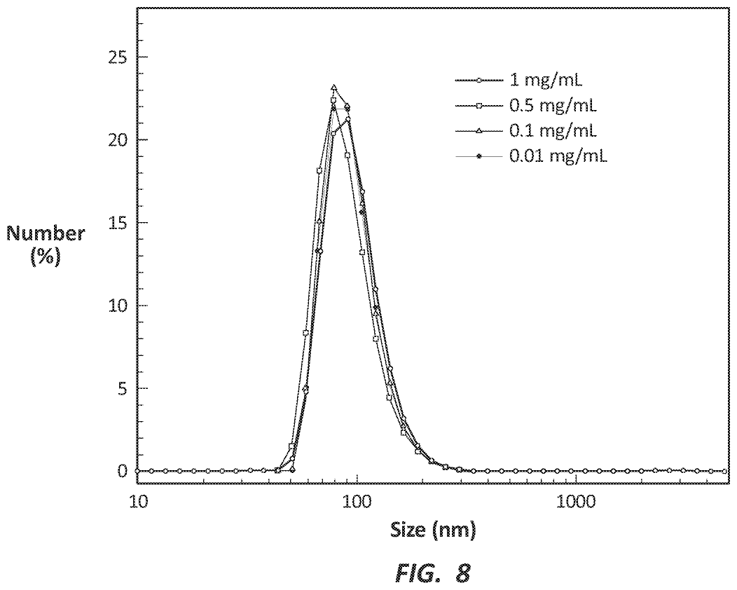

Careful optimization of the F127 polymer concentration in the coating solution facilitates formation of nanoparticles with high echogenicity and good dispersibility in buffer solutions. While using lower F127 concentrations increases the ultrasound responsiveness of the particles, below a particular concentration, the F127 amount is not sufficient to stabilize particles; thus, they coagulate in buffer solutions. On the other hand, relatively high F127 concentrations may yield particles with poor ultrasound responsiveness (FIG. 6). In initial experiments, the effect of the F127 amount in the coating solution was studied by stabilizing hMSN in PBS using different F127 concentrations of between 0.25 and 2 mg/mL. Except for the sample prepared using 0.25 mg/mL F127, all samples were formed stable colloids in PBS. While dynamic light scattering (DLS) analysis demonstrated that all samples have good dispersibility in PBS with fairly narrow size distribution. It was observed that increasing the F127 amount resulted in narrower size distribution (FIG. 7A) with smaller polydispersity index (PDI) values (Table 1). Also, the tail of the peak around 200 nm, which is attributed to clumps of a few particles, disappeared at high F127 concentrations. In addition, the effect of dilution on their dispersibility in PBS was explored since the particles were stabilized by F127 through physical interactions. The sample prepared using 0.5 mg/mL F127 polymer was serially diluted in PBS from 1 mg/mL to 10 micrograms per milliliter (μg/mL), and DLS analysis was performed (FIG. 8). Even at the lowest particle concentration, F127 stabilized hMSN demonstrated a very similar size distribution with the as-prepared sample (1 mg/mL). In the first aspect, the amphiphilic molecules are spaced apart from one another on the silane layer by distances configured to provide bubble nucleation sites that will initiate cavitation of echogenic bubbles in response to acoustic intensity with a mechanical index of about 1.9 or less, or about 1.5 or less, or about 1.3 or less or about 1.0 or less, or about 0.7 or less.

TABLE 1
Poly dispersity index values of hMSN dispersed
in PBS using different amounts of F127.

| F127 amount (mg/mL) | PDI |
|---|---|
| 0.25 | 0.218 ± 0.0032 |
| 0.375 | 0.169 ± 0.0142 |
| 0.5 | 0.057 ± 0.0081 |
| 1 | 0.045 ± 0.0088 |
| 2 | 0.03 ± 0.0074 |

The stabilized gas-pockets (i.e., nanobubbles) at the hydrophobic interface of F127-hMSN can nucleate echogenic microbubbles under reduced acoustic pressures and, thus, enhance the contrast of the ultrasound images. Accordingly, the ultrasound responsiveness of F127-hMSN samples was tested using an IP-105 linear array transducer (Sonic Concepts). For ultrasound imaging, 2 mL of samples (100 μg/mL in PBS) was placed in the bulb of a plastic transfer pipette, and B-mode images were collected at an imaging frequency of 2.5 MHz and a MI of 1.4. FIG. 7B inset shows the typical B-mode images of the F127-hMSN samples, where the generated bubbles can be observed as bright spots, especially for the samples prepared using lower F127 amounts. On the other hand, there was no contrast enhancement in the absence of particles (FIG. 7B inset; PBS). To quantify the ultrasound contrast enhancement, 1-second-long videos (100 frames/second) were analyzed. For each video frame, the intensity in the region of interest was calculated using Image J software, and the intensities of each frame were averaged. FIG. 7B shows the average intensities generated by the F127-hMSN samples (100 μg/mL in PBS). Strong ultrasound contrast enhancement was achieved at F127 concentrations between 0.25 and 0.5 mg/mL, and a reduction in the generated intensity was observed with increasing F127 concentrations. In addition, non-functionalized MSNs with hydrophilic surfaces or F127 polymer solution (0.05 mg/mL, which equals to the F127 concentration of the sample prepared using 0.5 mg/mL F127 after dilution for imaging) in the absence of any particles were tested as controls. For both samples, the signal was not significantly different from the PBS background, which suggests that a hydrophobic interface is required to generate echogenic bubbles. Based on the DLS and US imaging results, 0.5 mg/mL of F127 concentration was found to yield contrast agents with high echogenicity and good dispersibility in PBS and, thus, used in further testing described below unless otherwise specified.

FIG. 7C shows the ultrasound contrast enhancement at different F127-hMSN concentrations between 0-250 μg/mL. Even at the lowest particle concentration, 2.5 μg/m L, F127-hMSN produced an ultrasound signal that was approximately 3-fold more intense than the background signal. At low particle concentrations up to 10 μg/mL, the ultrasound signal increased almost linearly. At higher concentrations, on the other hand, the increase in the average signal intensity was less pronounced. Therefore, the stabilizer amount is preferably selected to maximize the ultrasound contrast without affecting the dispersibility of contrast agents in buffer solutions. This was performed for F127 coating of different types of hydrophobically modified silica nanoparticles including, 50 nm mesoporous silica nanoparticles, large pore dendritic silica nanoparticles, silica nanocups, and solid silica nanoparticles. In an embodiment of the first aspect, the amphiphilic stabilized hydrophobic nanoparticles are used at concentrations of about 2.5 micrograms per milliliter up to about 100 micrograms per milliliter. In the embodiment of the second aspect, the amount of amphiphilic molecule added to a dried film of nanoparticles is a quantity configured to facilitate binding of the amphiphilic molecules to the hydrophobic nanoparticles spaced-apart by a distance that allow cavitation of echogenic bubbles in response to acoustic intensity at mechanical index of about 1.9 or less, or about 1.5 or less, or about 1.3 or less or about 1.0 or less, or about 0.7 or less. In an embodiment of the second aspect, the amphiphilic stabilized hydrophobic nanoparticles that are capable of being imaged by ultrasound equipment delivering acoustic intensity at a mechanical index of about 1.9 or less, or about 1.5 or less, or about 1.3 or less or about 1.0 or less, or about 0.7 or less are prepared with an amount of the amphiphilic molecule being in the range of about 1:2 to 1:4 in milligrams when compared to the amount of dried nanoparticles. In other words, the ratio of the amount of the amphiphilic molecule to the amount of the dried film of nanoparticles may be 1:2, 1:4, or any ratio in between, for example 1:3.

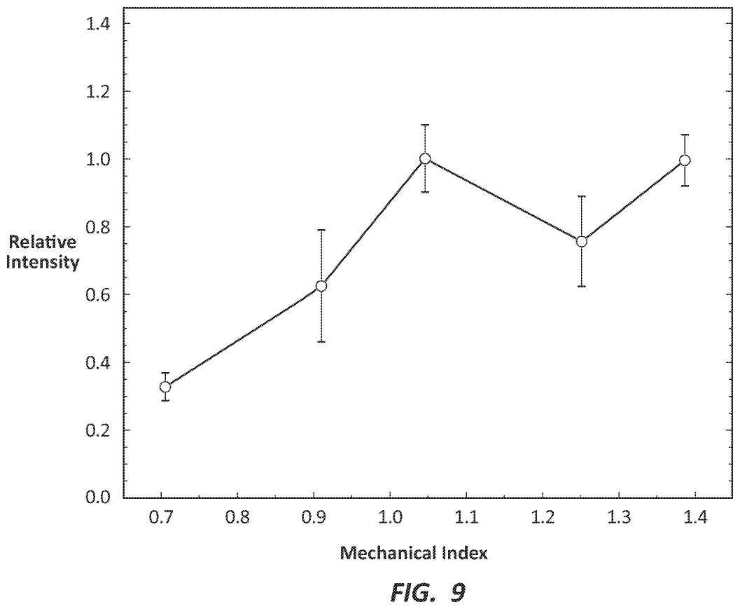

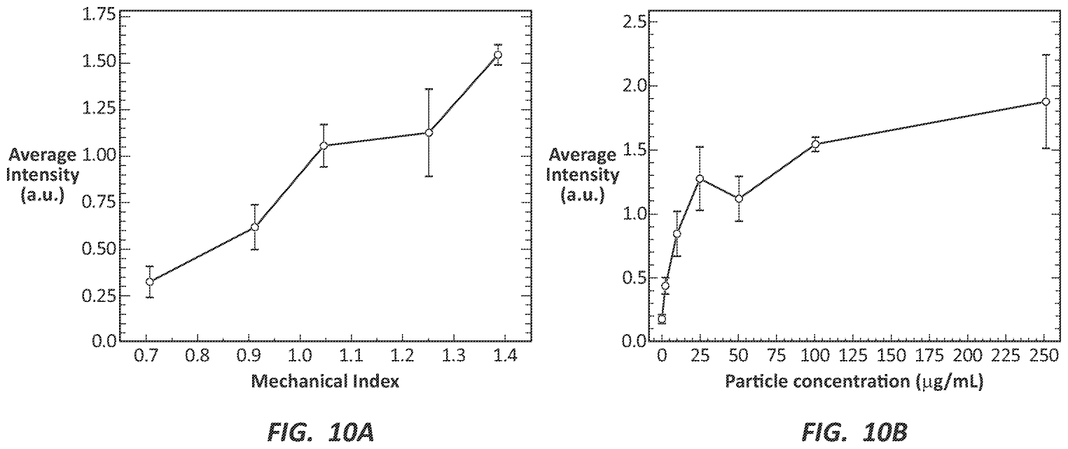

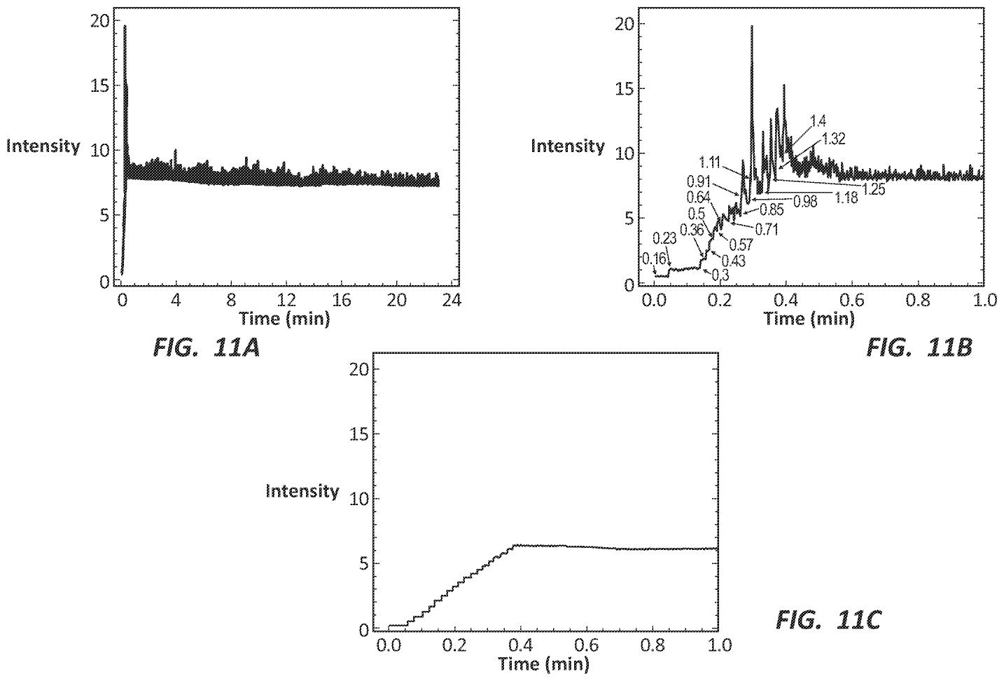

F127 coated hydrophobic nanoparticles were observed to improve the contrast of the ultrasound images (which were collected using an imaging transducer operating at 2.5 MHz) at mechanical indices as low as 0.7 (FIGS. 7B and FIGS. 9 and 10A), which is well below the FDA limit of 1.9, and available on conventional medical ultrasound instruments. Generation of bubbles was observed at MI values as low as 0.7, and the average intensity was increased gradually up to MI of ˜1.05. The generation of more bubbles at higher MI values can be observed during gradually increasing the imaging MI. In addition, the nanoparticles produced ultrasound contrast at low particle concentrations down to 2.5 μg/mL (FIG. 10B). Moreover, it was shown that the ultrasound contrast agents can produce echogenic bubbles for at least 20 min under continuous ultrasound imaging (FIGS. 11A-11C).

To further explore the ultrasound contrast generation ability of F127-hMSN and its durability, videos were recorded at different imaging conditions. First, the MI was rapidly increased from 0.2 to 1.4 and the intensity was measured in the region of interest of each video frame (FIG. 7D). When the MI was suddenly increased to 1.4 at around 0.3 seconds, a burst increase in the intensity was observed with the generation of numerous bubbles or “bubble cloud” formation (FIG. 7D middle inset). The initial signal decreased rapidly and reached a plateau at around 1 second, where bubble generation was still clearly observable, although less frequent compared to the initial signal. In addition, it was observed that the rapid increase in signal could be achieved for at least 5 times from the same F127-hMSN sample by turning the imaging probe off and on again. Next, to investigate the stability of the signal, signal generation by F127-hMSN was recorded under continuous imaging for 20 min. Here, MI increased gradually from 0.2 to 1.4 in the first ˜30 seconds and kept constant for the next ˜20 min. Similarly, bubble cloud formation was observed at MI values higher than 0.7 as rapid increases in the intensity (FIGS. 11A AND 11B). It should be noted that in the absence of nanoparticles, rapid increases in the intensity plot were not observed (FIG. 11C). Importantly, the generation of bubbles could still be observed even after 20 min of continuous imaging (FIG. 11A).

It should be noted that ultrasound contrast generation by F127 polymer stabilized hydrophobic mesoporous silica nanoparticles have been demonstrated in another study. However, the nanoparticles that were prepared in this study cannot be imaged in the absence of HIFU as they require high acoustic intensities (PNP of 9.87 MPa, and MI of 9.4) to generate bubbles. In contrast, the particles that are developed in the current study can generate echogenic bubbles at around an order of magnitude lower PNP and MI values; 1.1 MPa and 0.7, respectively, which falls in the pressure range of conventional medical instruments. Thus, they can be imaged using the ultrasound imaging systems that are readily available at hospitals without the need for any additional instrumentation for HIFU insonation.

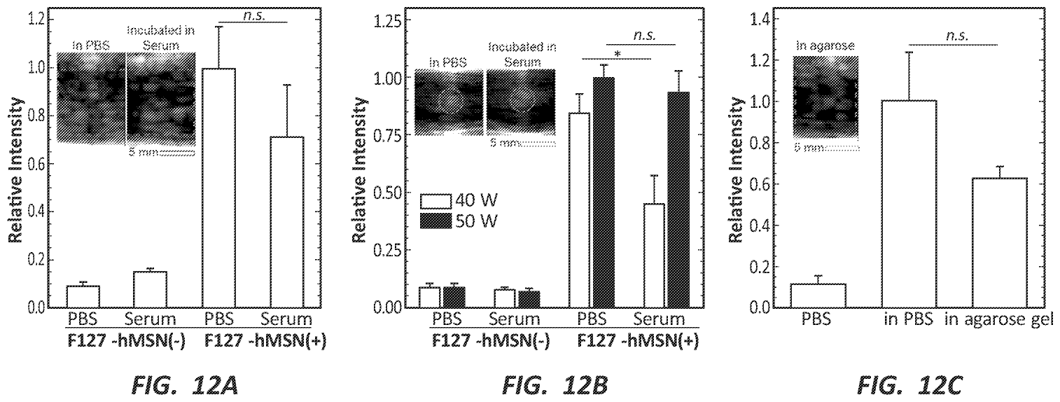

To examine the potential of amphiphilic stabilized hydrophobic nanoparticles as contrast agents for in vivo ultrasound imaging, the ultrasound responsiveness of the particles was tested after incubating them in 50% fetal bovine serum (FBS) at 37° C. for overnight using both the imaging probe and the HIFU transducer (FIGS. 12A and 12B). There was a slight decrease in the average signal (˜30%) generated by the F127-hMSN after serum incubation when insonated using the imaging probe, although the decrease was not statistically significant (FIG. 12A). For the HIFU insonation, while there was a statistically significant decrease in the signal (˜50%) after serum incubation at 40 W, the decrease was negligible at 50 W (FIG. 12B). The slight decrease in the signal may be due to dissolution of some gas-pockets due to the protein adsorption to the nanoparticle surface. The responsivity of F127-hMSN in tissue-mimicking phantoms using the imaging probe was also tested (FIG. 12C). F127-hMSN containing agarose gels were prepared by mixing the particles with warm agarose (˜40° C.) to give final particle concentration of 100 μg/mL and agarose concentration of 1% (w/w). After allowing the gels to solidify at RT, the phantoms were imaged (MI=1.4), as described above. The signal generated by F127-hMSN in agarose phantoms was clearly distinguishable from the background (˜5 fold more intense) (FIG. 12C). Although the generated intensity was reduced by ˜35% compared with the intensity generated in PBS, the decrease was not statistically significant. Overall, the results presented in FIGS. 12A-12C indicate the potential of F127-hMSN in nucleating the growth of micron-sized bubbles in complex biological environments.

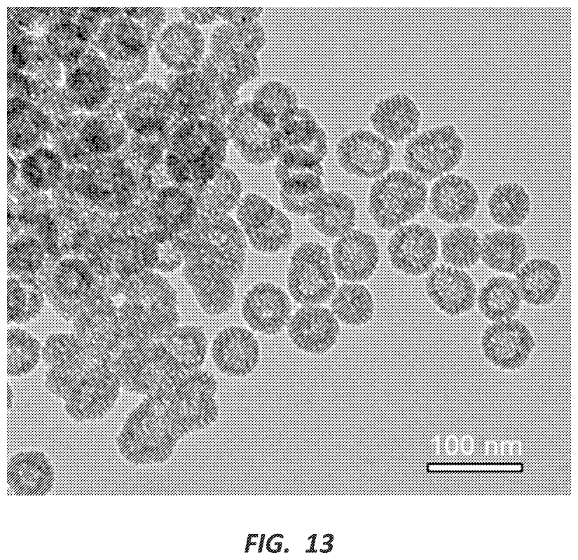

Preferably, contrast agents should be substantially completely degraded in the body after injection in reasonable time frame (less than 1 month, for example, in about 1 week, about 2 weeks, about 3 weeks). To examine the biodegradability of the contrast agents described here, the F127 coated hydrophobic MSNs were incubated in simulated body fluid (SBF) for 1 week at 37° C. The particles were mostly degraded after incubation (FIG. 13) such that it is expected that the particles would be degraded substantially completely under extended incubation in SBF or within the body of a subject. Nevertheless, this result indicates that the biodegradable contrast agents can be formulated. To achieve faster degradation, silica network can be modified (e. g. with the addition of organosilane monomers) or other types of biodegradable particles can be used. In some embodiments, the hydrophobic nanoparticle substantially completely biodegrades within a period of less than one month after introduction into a body of a subject.

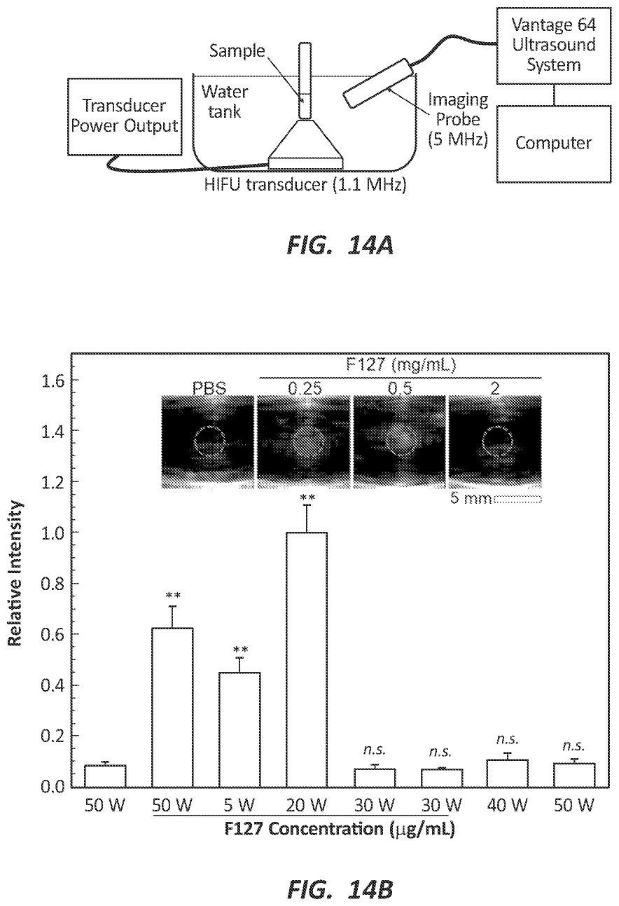

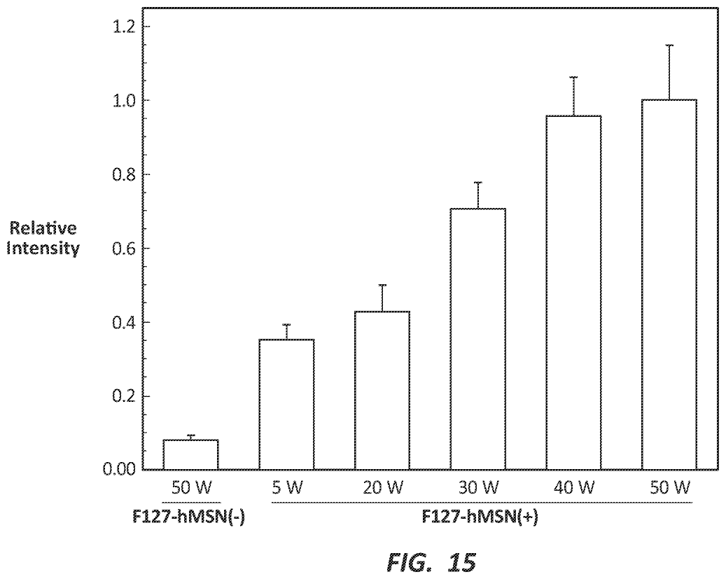

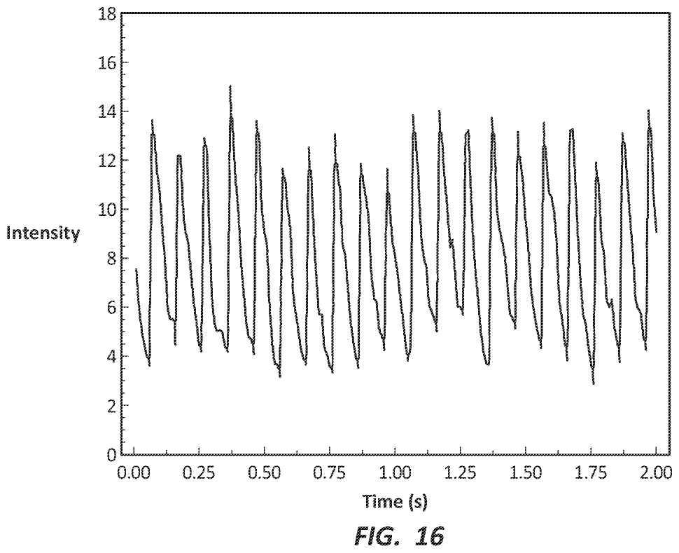

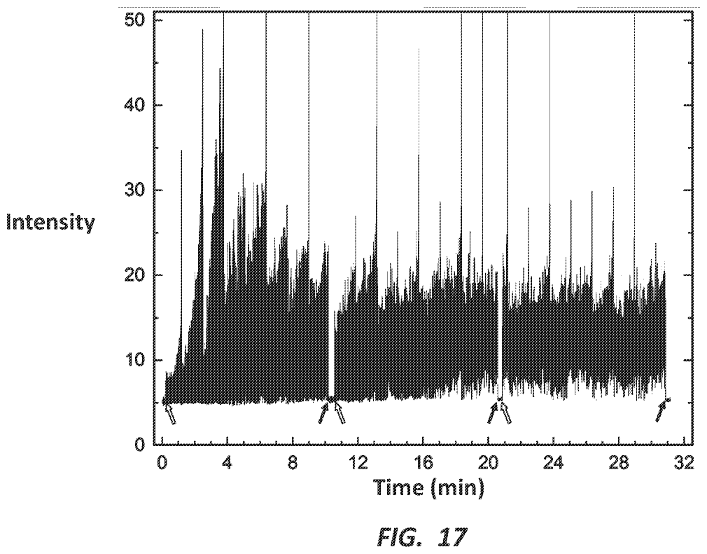

Beyond increasing the contrast of ultrasound images, these particles can also be applied to enhance the effects of therapeutic ultrasound. When the nanoparticle suspensions were insonated using HIFU, a technology that can be used for mechanical or thermal tissue ablation, blood-brain barrier opening, or local drug delivery, generation of acoustic cavitation events was also observed. The experimental set up is shown in FIG. 14A, where a 1.1 MHz HIFU transducer is used to insonate particles, and the generated microbubbles were imaged using a transducer operating at 5 MHz and MI of 0.26. For HIFU insonation, 10 microsecond (μs) ultrasound pulses with a pulse repetition frequency (PRF) of 10 Hz was used at 40 W transducer power input. The F127-hMSN samples (100 μg/mL in PBS) were imaged at low MI (0.26) to prevent bubble generation by the imaging probe in the absence of HIFU insonation. Similar to the experiments performed using the imaging probe, at higher F127 concentrations no bubble generation was observed and for the samples prepared using F127 concentrations between 0.25 and 0.5 mg/mL, the ultrasound signal was clearly distinguishable from the background (FIG. 14B). As expected, the signal was localized at the small focal zone of HIFU pulses. Also, bubble cloud formation after each HIFU pulse was clearly visible. In addition, the intensity generated by F127-hMSN was increased with the increasing electrical power input to the transducer (FIG. 15). As a result of bubble cloud formation, a sudden increase in the intensity after each HIFU pulse was observed in the signal intensity plot of the F127-hMSN (FIG. 16). No cavitation events were observed in the absence of F127 coated hydrophobic particles or the presence of unmodified silica nanoparticles. The stability of the HIFU responsiveness of the particles against protein adsorption by incubating the particles in serum as described above was also demonstrated (FIG. 12B). Importantly, F127-hMSN generated strong signal for at least 30 min under HIFU insonation, which indicates their durable cavitation generation ability (FIG. 17).

The preliminary results that are summarized here suggest that the amphiphilic stabilized hydrophobic nanoparticles can be used as nanoscale robust ultrasound contrast agents. As they generate ultrasound signal at low mechanical indices, they can be imaged using a variety of transducers (for example, linear, curvilinear, and phased array) operating at different frequencies (ranging, for example, from about 1.5 MHz to about 14 MHz, about 2 MHz to about 10 MHz, or about 5 MHz to about 12 MHz) and designed for different applications (e.g., abdominal, breast, thyroid, cardiovascular, transvaginal, transrectal, musculoskeletal or prostate imaging) that are readily available at hospitals. In addition, they can sensitize mechanical effects generated by HIFU treatment. Thus, they can also be used for improving the outcomes of ultrasound therapies including, mechanical tumor ablation, drug, gene, or nanoparticle delivery to solid tumors, blood-brain opening, sonoporation, sonodynamic therapy, and immunotherapy. Degradability and safety of these ultrasound contrast agents in in vitro experiments, and in vivo validation of the contrast agents in tumor imaging and therapy using animal models are being explored.

The high echogenicity of the F127-hMSN could be attributed to several reasons. Primarily, our results indicate that careful optimization of the F127 amount can enhance the acoustic activity of the F127 stabilized hydrophobic nanoparticles as a large amount of F127 at the nanoparticle surface could prevent nanobubble formation or cause destabilization (FIGS. 6 and 7B). Second, nanoparticle coating conditions (e.g., sonication, temperature, co-solvents) can affect the nanobubble nucleation and their dissolution. In particular, bath sonication (commonly used to disperse particles in aqueous solutions and to re-suspend them after centrifugation) may consume the surface nanobubbles as it is intense enough to nucleate cavitating bubbles. The stabilization methods described before for hydrophobic MSNs requires at least several minutes of bath sonication.

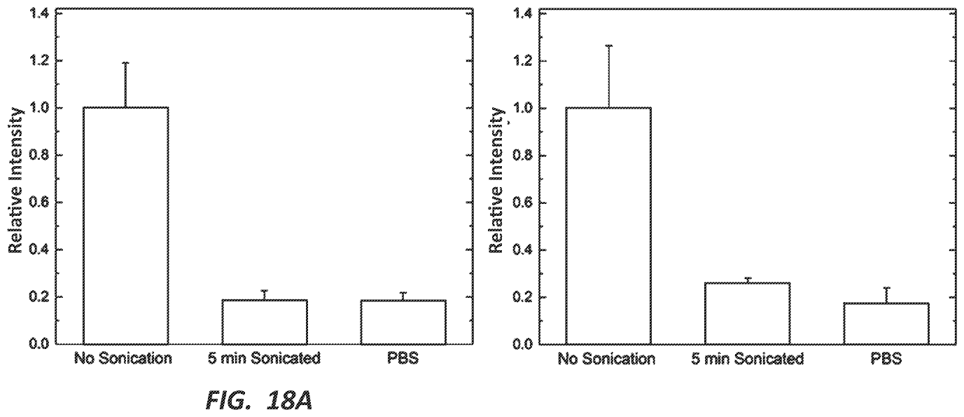

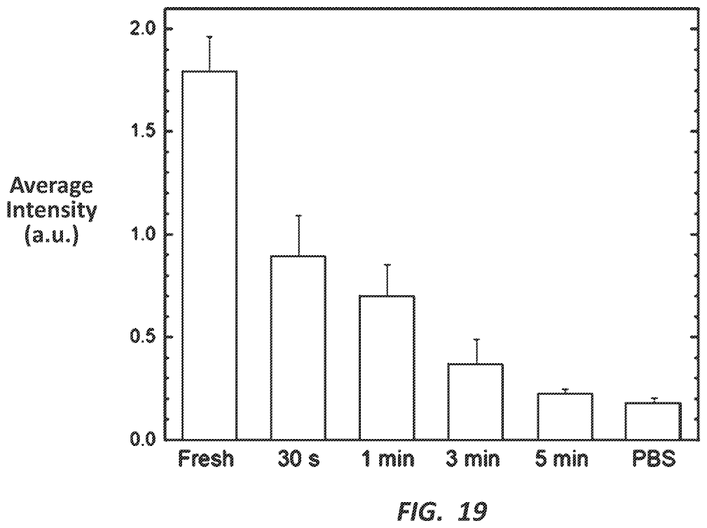

In contrast, the present method uses a brief bath sonication step (approximately 5 seconds) and no stirring, which enables their imaging using standard medical ultrasound instruments. Thus, it may be possible that the minimization of the sonication time results in the stabilization of more nanobubbles per particle. To demonstrate the deleterious effect of extensive bath sonication, which can consume the stabilized nanobubbles to generate acoustic cavitation, on contrast generation by the nanoparticles, nanoparticles were further sonicated for up to 5 min and imaged using an ultrasound transducer operating at 2.5 MHz or at 5 MHz. The 5 min bath sonication almost entirely decreased the ultrasound responsiveness of the nanoparticles (FIGS. 18A, 18B and 19). This result showed that the coating method enables ultrasound contrast generation by nanoparticles at low acoustic pressures, which falls in the pressure range of conventional medical instruments (about 0.1 MPa to about 4.0 MPa). On the contrary, the disclosed method can be applied to a variety of monodisperse particles with different sizes, morphologies, and shapes to prepare hydrophobic nanoparticles in gram scale (can potentially be scaled up to kilogram scale). The surfaces of the contrast agents can be easily modified. For example, the hydroxyl terminal groups of various poloxamers can be used for conjugating targeting agents (such as peptides, antibodies, nanobodies, aptamers, etc.). Similar chemistries can be applied to functionalize other types of amphiphilic molecules to modify contrast agent surfaces. Finally, some studies were directly applied nanoparticles with hydrophobic surfaces without any amphiphilic coating. While such nanoparticles have strong ultrasound responsiveness, without a biocompatible hydrophilic stabilization layer, they can aggregate in biological solutions and cause severe acute toxicity. Lastly, the physical and chemical properties of the nanoparticles, such as particle size, shape, hydrophobicity, and surface roughness, can affect the acoustic of nanoparticles and activation threshold and can be studied to improve the ultrasound responsiveness of F127-hMSN further.

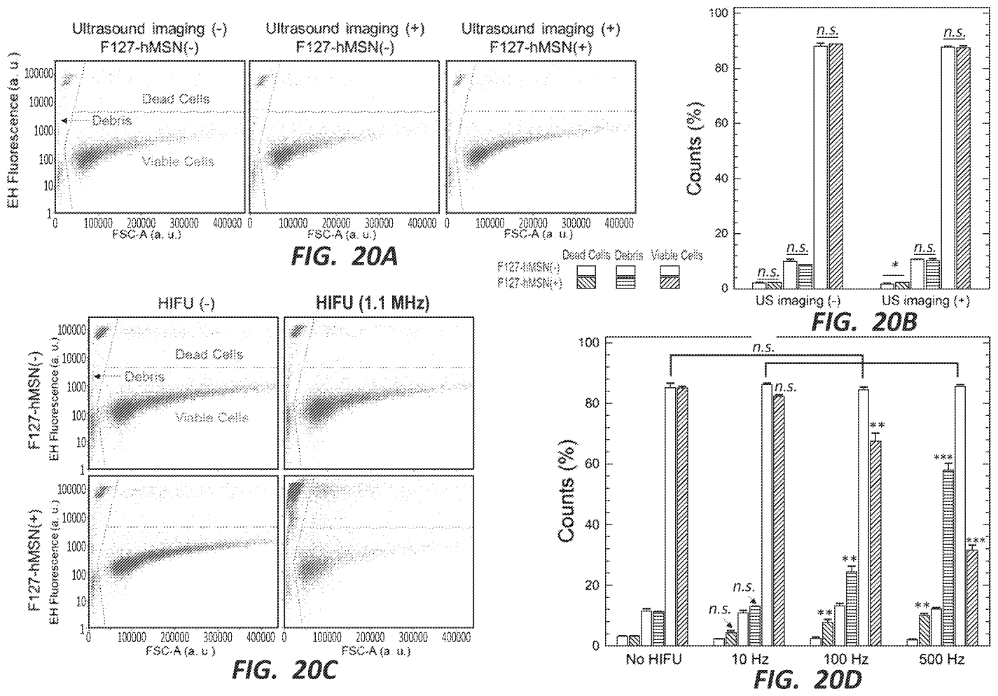

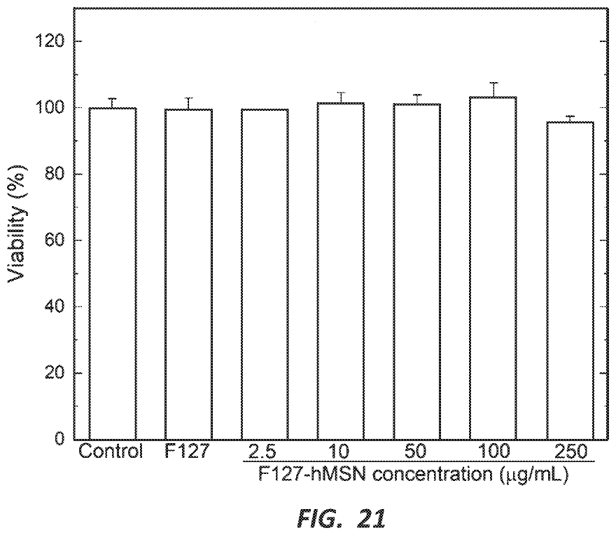

The acoustic cavitation events generated by F127-hMSN can potentially induce damage to the cells or tissue. While this is desired for therapeutic purposes, such as histotripsy or drug delivery, it is an unwanted effect in ultrasound imaging applications. Thus, the potential bioeffects of F127-hMSN under imaging or HIFU conditions were evaluated using 4T1 murine mammary carcinoma cells. F127-hMSN was mixed with 4T1 cells (10⁶ cells/mL in RPMI-1640) in a bulb of a plastic pipette and exposed to ultrasound for 2 min. In imaging experiments, 1 mL of cell suspensions were imaged with the probe operating at 2.5 MHz (MI=1.4). Then, live and dead cells were labeled fluorescently, and flow cytometry was performed. As cell ablation produces cell debris, the debris in the flow cytometry scatter plots were also included in the analyses. FIG. 20A shows the representative scatter plots of forward scatter-area versus the fluorescence of ethidium homodimer-1 (EH, dead cell stain), where three distinct populations can be observed; i) debris, ii) viable cells, and iii) dead cells. Also, in the debris region, two sub-populations with or without EH staining were observed. However, for the sake of simplicity in the analysis, they were considered as a single population. FIG. 20B shows the percentage of events recorded for all three populations in the presence or absence of F127-hMSN and with or without ultrasound imaging. The percentages of the events recorded in the three regions were very similar, with no statistically significant difference for most of the cases. There was only a small increase in the number of dead cells compared to control from 1.9%±0.5 to 2.7%±0.3 when they were insonated in the presence of F127-hMSN. For the same sample, the changes in the two other regions were not statistically significantly different from the control. Overall, these results indicated that the cavitation events generated by F127-hMSN under imaging conditions were generally safe and caused less than 1% cell death. Also, flow cytometry analysis demonstrated that the F127-hMSN did not show any intrinsic cytotoxicity in the absence of ultrasound insonation. The good cytocompatibility of F127-hMSN was further verified using the MTS assay (FIG. 21).

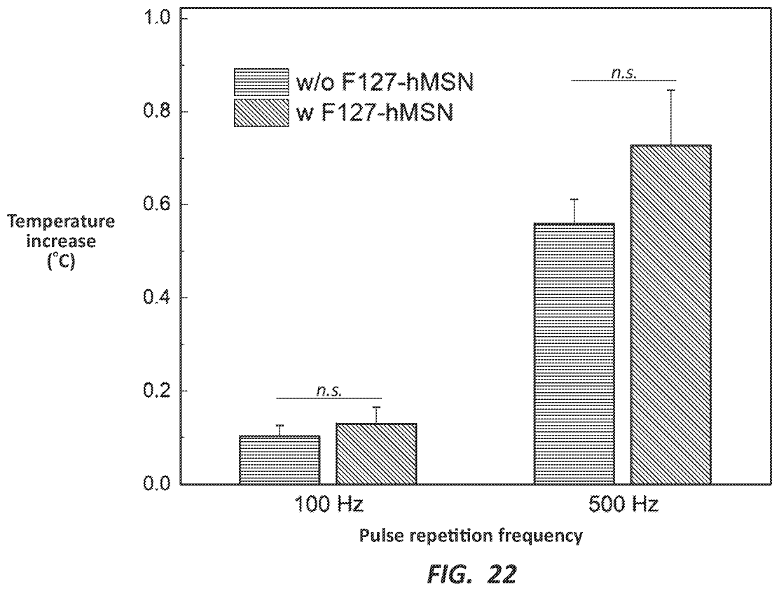

When the HIFU transducer was used to generate cavitation events, on the other hand, abundant cell ablation and death were observed. For HIFU insonation, 0.5 mL of samples were exposed to ultrasound (1.1 MHz) for 2 min at 50 W transducer power input, pulse duration of 10 μs, and PRF of 10, 100, or 500 Hz. While there were statistically non-significant changes in the percentages of cell viability, dead cells, and cell debris at PRF of 10 Hz, the changes in these regions became more pronounced at higher PRF, where viability decreased to around 35% at 500 Hz (FIGS. 20C and 20D). Importantly, in the absence of F127-hMSN, there was no statistically significant change in the cell viability even at the highest PRF of 500 Hz, indicating that the cavitation events nucleated by F127-hMSN caused the cell death/ablation. The temperature increase in the cell suspension after HIFU insonation in the presence or absence of F127-hMSN was also measured (FIG. 22). Even at the highest pulse repetition frequency of 500 Hz, the temperature increases were only around ˜0.7° C., which indicates that the cavitation events generated by F127-hMSN mainly killed the cells. Also, the temperature increase at 10 Hz was undetectable, and it was barely detectable at 100 Hz (˜0.1° C.). In addition, the presence of F127-hMSN did not result in a significant change in the temperature increase compared with samples without nanoparticles. Overall, the results presented demonstrate that F127-hMSN could be used for both imaging and therapeutic purposes by simply adjusting the insonation conditions.

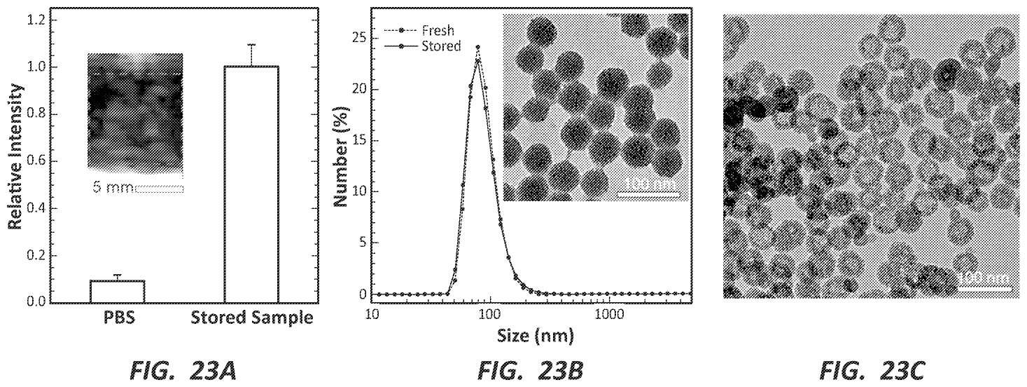

Next, the stability of the particles during storage was explored. FIG. 23A shows the ultrasound imaging (2.5 MHz, MI of 1.4) of the F127-hMSN sample (100 μg/mL in PBS) after storing at RT for 1 month at a particle concentration of 1 mg/mL in PBS. F127-hMSN exhibited a similar increase in the ultrasound signal after storage, demonstrating the stability of the nanobubbles against dissolution. Also, no particle degradation or change in the particle morphology at the storing conditions were observed (FIG. 23B inset). In addition, DLS analysis of the stored sample demonstrated that the size distribution of the F127-hMSN did not change during storage (FIG. 23B).

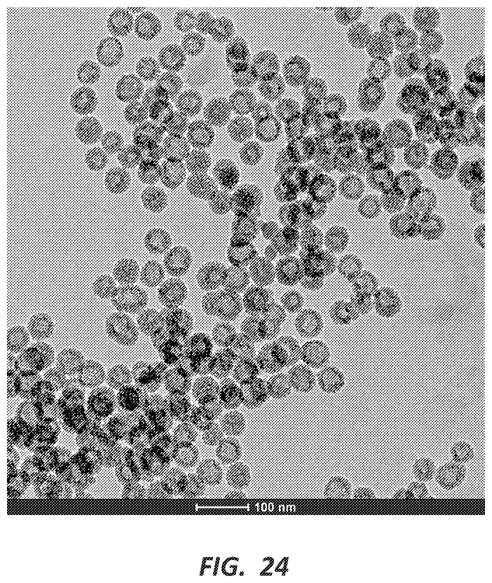

Finally, the degradation of F127-hMSN in simulated body fluid (SBF) was studied. Silica nanoparticles can degrade in aqueous solutions overtime through the hydrolysis of siloxane bonds by water molecules. It is well known that at high particle concentrations (>0.1 mg/mL), MSNs are resistant against dissolution in buffer solutions. However, when they are diluted, such as after intravenous injection, they can be dissolved in from several hours to months depending on the properties of the silica network (e.g., crosslinking density, presence of comonomers). While one can expect that the hydrophobic surface modification of F127-hMSN can block water penetration into the particles and thus prevent particle degradation, significant silica dissolution for diluted the F127-hMSN sample (50 μg/mL) was observed after stirring at 37° C. in SBF for 1 week with the formation of hollow particles (FIG. 23C). Indeed, this type of degradation is typical for MSNs; degradation starts from inside of the particles, followed by the formation of silica shells, and finally complete dissolution. Further incubating the particles in SBF for an additional 3 weeks did not noticeably affect the degradation of the particles (FIG. 24). While the tested particles demonstrated a rather slow degradation, their degradation properties may be improved by simply changing the silica network, such as the addition of an organosilane monomer during synthesis to decrease the crosslinking density of silica network.

The method described here enables preparation of robust nanoscale ultrasound contrast agents in large scales and by using different types of particles (10-200 nm) and stabilizers. The contrast agents can be imaged using standard medical ultrasound instruments at low particle concentrations. In addition, they can be used to enhance the effects of therapeutic ultrasound. Furthermore, their surfaces can be modified for targeting them to tumors, and they can be formulated to be biodegradable.

The disclosed is a method to prepare novel GSNs (F127-hMSNs) that combines high ultrasound responsiveness with small particle size. The amphiphilic stabilized hydrophobic nanoparticles can be prepared using hydrophobically modified ˜50 nm mesoporous silica nanoparticles and a biocompatible amphiphilic copolymer (e.g., F127). The F127-hMSNs demonstrated excellent dispersibility in buffer solutions with average sizes smaller than 100 nm. Unlike the GSNs prepared in previous studies, which requires HIFU insonation for the generation of echogenic bubbles, the F127-hMSNs can be imaged using a conventional ultrasound imaging probe in PBS, serum or agarose gels at mechanical indices as low as 0.7. Also demonstrated is that the F127-hMSNs can be continuously imaged for at least 20 min and at low particle concentrations down to 2.5 μg/mL. In addition, they can be stored at room conditions for at least a month without any loss in their ultrasound responsiveness. Furthermore, the degradation of F127-hMSN in simulated body fluids at 37° C. was shown, which suggests the biodegradation potential of the F127-hMSN in vivo. The good safety profile of the ultrasound imaging process using F127-hMSN at low acoustic intensities was demonstrated using flow cytometry. On the other hand, insonating the particles using a HIFU transducer at higher acoustic intensities, produced strong cavitation activity to ablate the cancer cells effectively. Taken together, these results reveal that F127-hMSNs can be used for both imaging and therapeutic purposes. Thus, the GSNs described here may be utilized in the clinic for several applications, including molecular ultrasound imaging of solid tumors, drug delivery, and cancer therapy.

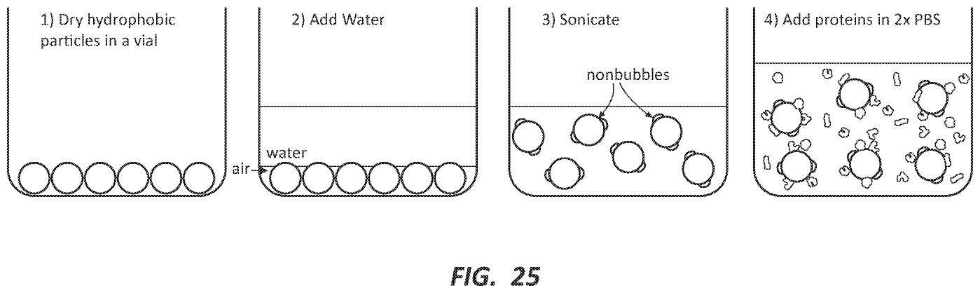

The inventors have found that, unexpectedly, nanoparticles that are functionalized with a reduced amount out hydrophobic monomer exhibit enhanced biodegradability, particularly when stabilized with proteins. Accordingly, in embodiments of the first aspect, stabilized hydrophobic nanoparticles having enhanced biodegradability can comprise proteins as stabilizing molecules. FIG. 25 depicts steps of preparing the protein-coated nanoparticles according to another embodiment of this disclosure. For protein stabilization, first, the hydrophobic particles are dispersed in a suitable organic solvent (chloroform, ethanol, acetone etc.), and dried in a vial (glass, plastic, metal vials with different sizes and shapes). Unlike the amphiphilic polymer-coated nanoparticles, the nanoparticles modified using a reduced amount of hydrophobic monomer can be directly dispersed in water by brief bath sonication (around 5 seconds) without adding an amphiphilic molecule such as F127. However, when these nanoparticles are dispersed in buffer solutions such as PBS, they can aggregate due to the formation of salt bridges between nanoparticles. Thus, albumin (or other protein sources such as serum or plasma) can be added to the PBS to prevent nanoparticle aggregation. In a typical procedure, 1 mL of hydrophobic nanoparticle dispersion (4 mg/mL in ethanol) is placed in a 20 mL glass vial and the ethanol is evaporated at 65° C. to form a thin film of nanoparticles. The dried films are further kept at 120° C. to remove any water or other solvents absorbed by the nanoparticles. Alternatively, particles can be kept under a vacuum to remove any adsorbed solvents. Then the nanoparticles are suspended in 2 mL of deionized water with a brief (around 5 seconds) bath sonication. Finally, particles are mixed with 2 mL of bovine serum albumin solution (1-50 mg/mL) in 2×PBS (20 mM, pH 7.4) and incubated at room temperature for 1-2 h to allow the formation of protein corona around nanoparticles. Using this protocol, nanoparticles with excellent acoustic activity and dispersibility in PBS can be prepared.

Proteins obtained from one or a combination of different sources such as human or animal plasma or serum, milk (lactalbumin, lactoferrin, 3-lactoglobulin, whey protein concentrates, whey protein isolates, casein, etc.) egg (ovalbumin, conalbumin, avidin, etc.), or soybean (soy protein isolates) or any recombinant protein can be used to coat and stabilize the hydrophobic nanoparticles. Non-limiting proteins of human serum origin that may be used include natural or recombinant alpha-globulin (including alpha-1-globulins and alpha-2-globulins), beta-globulin (including beta-1-globulins and beta-2-globulins), gamma-globulin, fibrinogens, hemoglobin, myoglobin, trypsin, chymotrypsin, etc. In some embodiments, the protein may comprise collagen or gelatin.

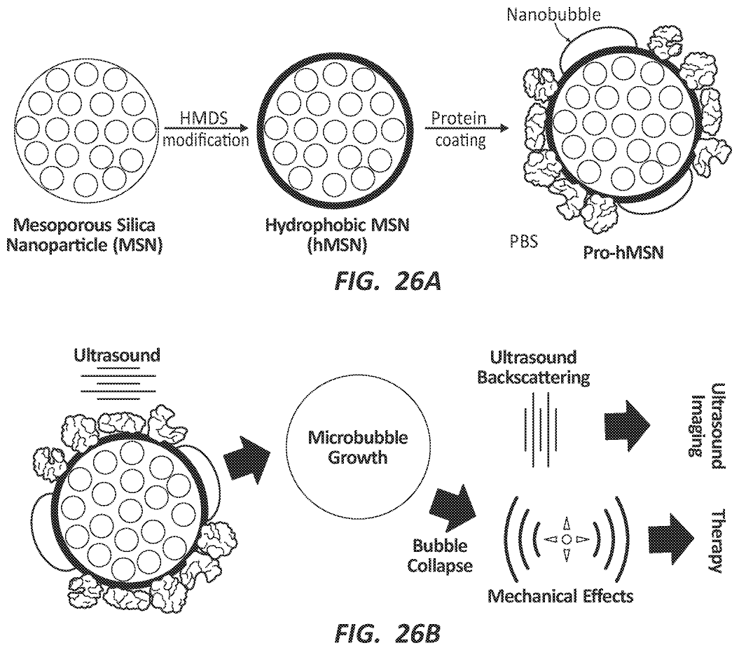

Protein coated MSNs can be used for ultrasound imaging and therapies. FIGS. 26A and 26B are a schematic representation of hydrophobic surface modification of mesoporous silica nanoparticles (MSNs) and their stabilization by proteins in buffer solutions (FIG. 26A), and microbubble generation by the nanoparticles under reduced acoustic pressures (FIG. 26B). The hydrophobic interfaces of the particles enable the stabilization of air-pockets at the particle surface, which can, in turn, nucleate acoustic cavitation events under reduced acoustic pressures, where echogenic micron-sized bubbles are generated. The violent collapse (i.e., acoustic cavitation) of these bubbles can generate mechanical effects in the tissue at high intensities. These bubbles can be used for both ultrasound imaging and therapies such as tumor ablation and drug delivery.

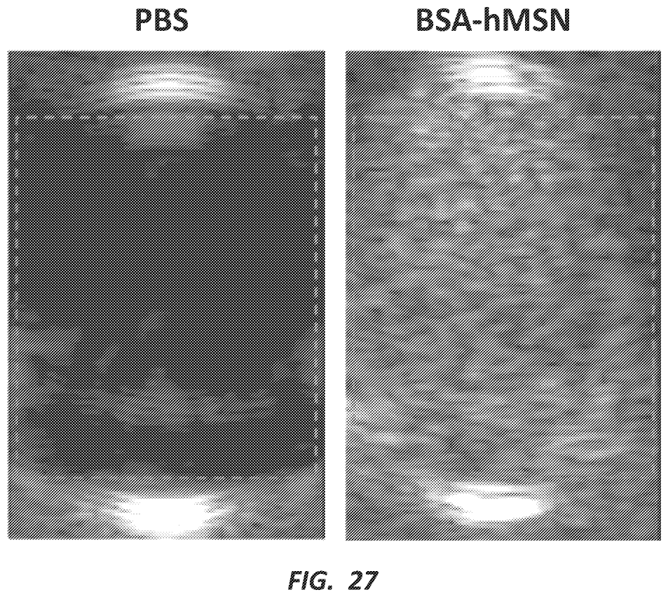

Protein-coated MSNs can be observed by ultrasound imaging. FIG. 27 shows representative ultrasound images showing contrast generation by bovine serum albumin (BSA) coated hydrophobic MSNs (BSA-hMSN). Ultrasound images were recorded using an imaging transducer operating at 2.5 MHz at a mechanical index of 1.5, which is below the FDA limit (1.9) and available on conventional medical ultrasound instruments.

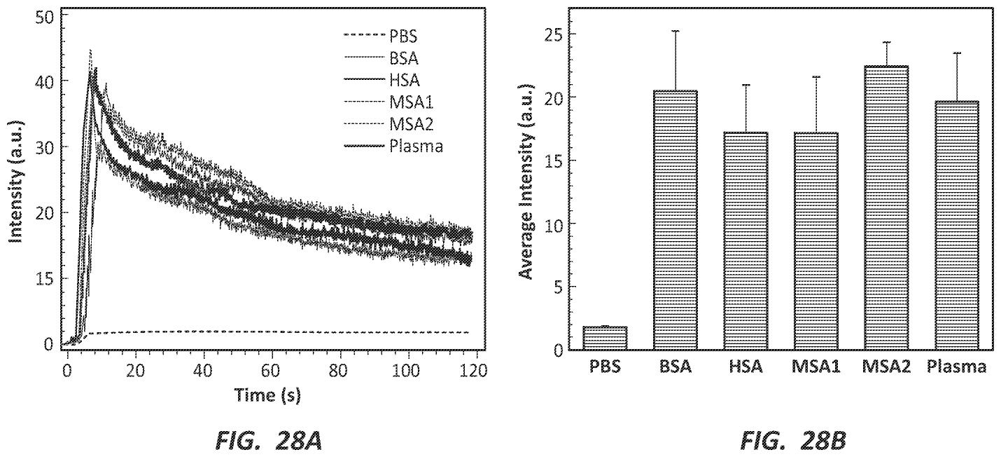

Hydrophobic MSNs coated with PBS, bovine serum albumin (BSA) purified using heat shock method, recombinant human serum albumin (HSA), mouse serum albumin (MSA1), mouse serum albumin produced by cold alcohol fractionation (MSA2), or pooled human plasma (plasma), were video recorded under continuous ultrasound imaging for 2 min. Videos were recorded using an imaging transducer operating at 2.5 MHz at a mechanical index of 1.5, which is below the FDA limit (1.9) and available on conventional medical ultrasound instruments. FIG. 28A shows (a signal intensity plot of hMSN (100 μg/mL) suspensions in PBS (10 mM, pH 7.4) stabilized using different protein sources (5 mg/mL) under continuous ultrasound imaging (2.5 MHz) for about 2 minutes. MI was gradually increased from 0.2 to 1.45 in the first approximately 10 seconds. FIG. 28B is a graph of average intensities generated by the particles. There was no statistically significant difference between different proteins suggesting that the acoustic activity of the nanoparticles is not dependent on the type of protein used for nanoparticle stabilization in buffer solutions.

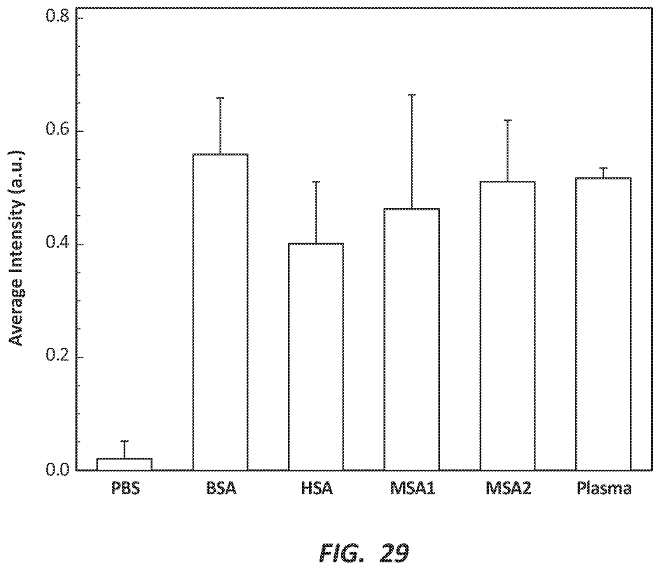

The particles described herein can be activated using a high intensity focused ultrasound (HIFU) transducer. FIG. 29 is a graph of average intensities generated by particles under HIFU insonation. A transducer operating at 1.1 MHz (power: 100 W, pulse duration: 20 us, pulse repetition frequency: 10 Hz) was used. There was no difference between the acoustic activities of nanoparticles coated with different proteins.

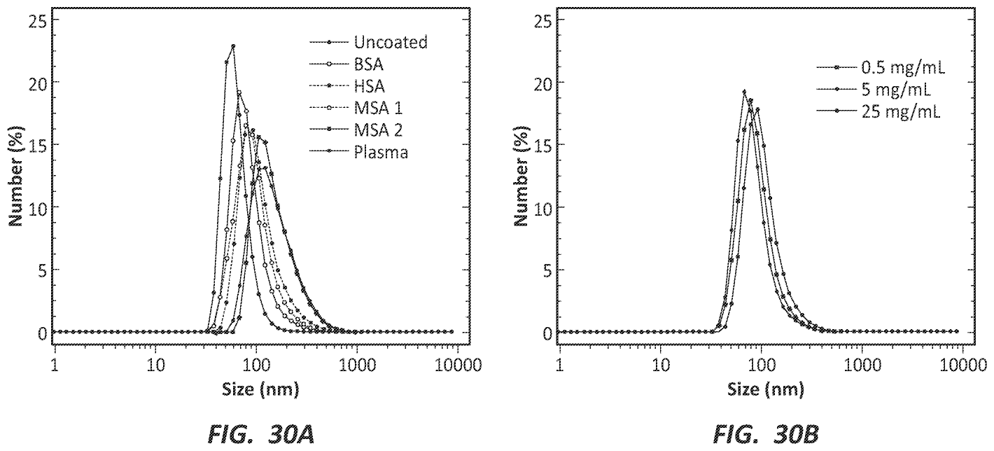

Size distributions of BSA, HSA, MSA1, MSA2, or plasma coated nanoparticles in PBS or uncoated particles in water were studied using dynamic light scattering (DLS) analysis. FIGS. 30A and 30B dynamic light scattering results of nanoparticles stabilized (FIG. 30A) using different proteins (5 mg/mL) and (FIG. 30B) using different amounts of BSA. Compared with uncoated nanoparticles, protein stabilized particles showed a slightly larger size distribution, indicating protein corona formation around the nanoparticles. The size distribution was similar for all of the proteins tested. The effect of protein concentration on size distribution in PBS was also studied (FIG. 30B). The DLS size distributions were similar for the particles stabilized with 0.5, 5, or 25 mg/mL BSA. It was also found that the protein concentrations below 0.5 mg/mL were not sufficient to prepare stable suspensions in PBS.

The stabilizing molecule solution used to prepare the stabilized hydrophobic nanoparticle for an ultrasound imaging contrast agent may have a protein concentration as low as about 0.5 mg/mL, a protein concentration from about 0.5 mg/mL to 100 mg/mL, from about 0.5 mg/mL to about 75 mg/mL, from about 0.5 mg/m L to about 60 mg/mL, or from about 1 mg/m L to about 50 mg/m L.

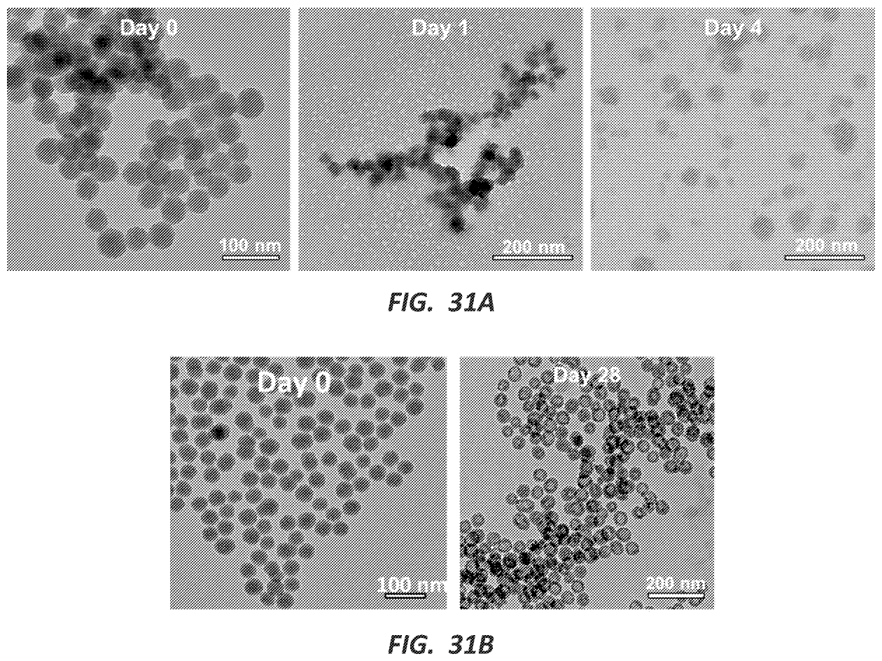

FIGS. 31A and 31B show transmission electron microscopy images of BSA coated hydrophobic MSNs over time. In simulated body fluid, complete degradation of BSA coated hydrophobic MSNs showed complete degradation in 4 days (FIG. 31A). In contrast, the nanoparticles coated in F127 polymer only partially degraded even after 4 weeks of incubation at the same experimental conditions (FIG. 31B).

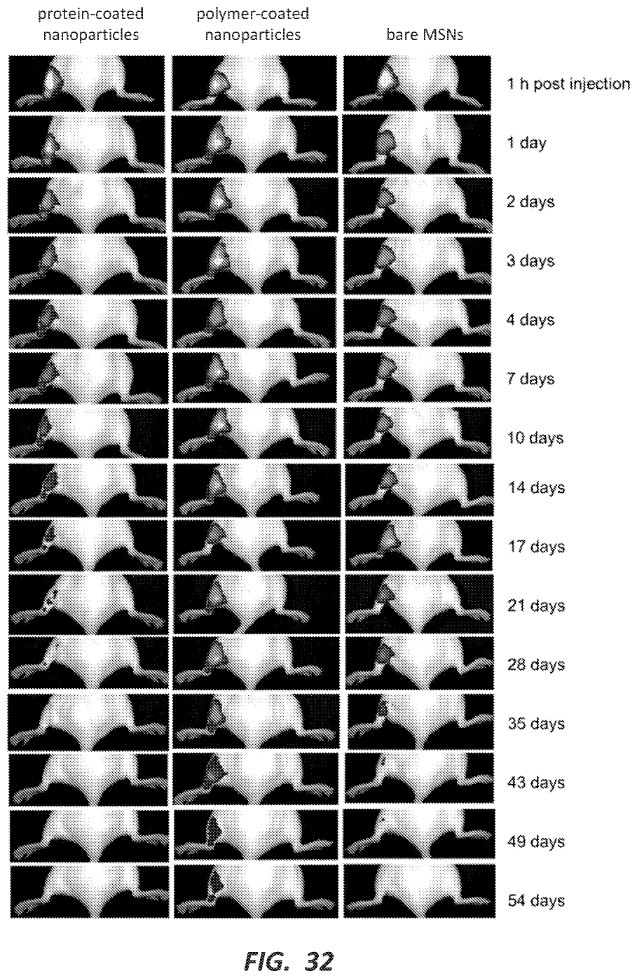

FIG. 32 shows in vivo imaging of mice that received intramuscular injection of protein-coated nanoparticles (left panels), F127 polymer-coated nanoparticles (center panels), or bare MSNs (right panels) over 54 days. It was observed that the fluorescence of protein-coated nanoparticles and bare MSNs quickly decreased in the first few days, indicating that they started to degrade. Protein-coated nanoparticles almost completely degraded by 4 weeks. On the other hand, the fluorescence of F127-coated nanoparticles remained for more than 4 weeks indicating their slower degradation.

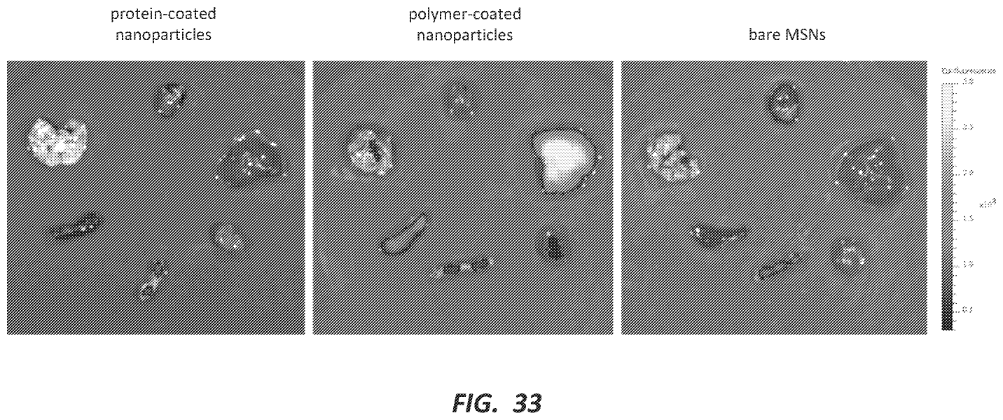

FIG. 33 shows fluorescence images of various organs of mice that received protein-coated nanoparticles (left panel), F127-coated nanoparticles (center panel), or bare MSNs (right panel), 1 week after tail vein injection. The fluorescently labeled nanoparticles (200 μL, 10 mg/mL in saline) were injected into the mice. No acute toxicity was observed at this nanoparticle dose (100 mg/mL), indicating the biocompatibility of the nanoparticles. Animals were sacrificed after 1 week, and the organ distribution of the nanoparticles was detected using an in vivo imaging system (IVIS). Almost no fluorescence signal was detected for protein-coated nanoparticles and bare MSNs, indicating their almost complete degradation. For F127-coated nanoparticles, a strong fluorescence signal was observed in the liver.

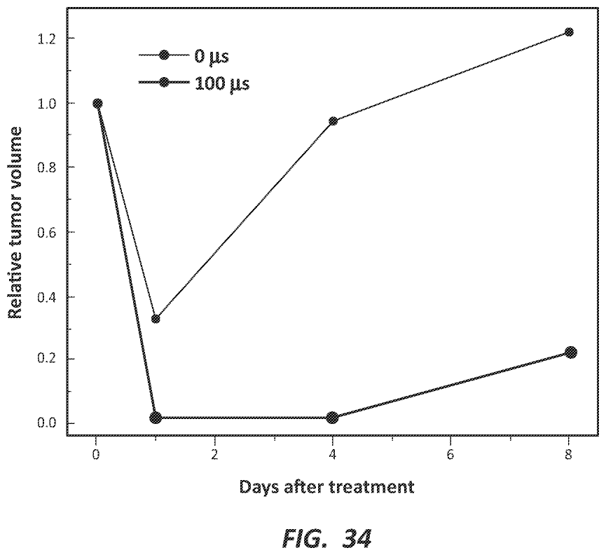

In vivo studies showed that both amphiphilic molecule-stabilized nanoparticles and protein-stabilized nanoparticles can be used to ablate solid tumors under HIFU insonation. In some embodiments, a method of using the hydrophobic nanoparticles described herein in HIFU ablation therapy can comprise steps of a) delivering the hydrophobic nanoparticle to a target tissue at a concentration of about 0.05 mg/mL to about 10 mg/mL and b) insonating the target tissue with HIFU to reduce a volume of the target tissue. FIG. 34 is a graph of relative tumor volume over days after treatment with high-intensity focused ultrasound (HIFU) in mice bearing HCT-116 colon cancer xenografts with or without injection of mouse serum albumin (MSA)-coated hydrophobic MSNs. Two different pulse durations, 20 and 100 μs, were tested. Other HIFU conditions were the same for both treatments, where input power was 150 W and PRF was 500 Hz. BSA coated hydrophobic MSNs were intratumorally injected (100-200 μL, 1 mg/mL in saline) and HIFU treated immediately after injection. Reduction of tumor volume suggests that BSA coated hydrophobic MSNs can be used to mechanically ablate solid tumors under HIFU insonation for 1 min using a HIFU transducer operating at 1.1 MHz.

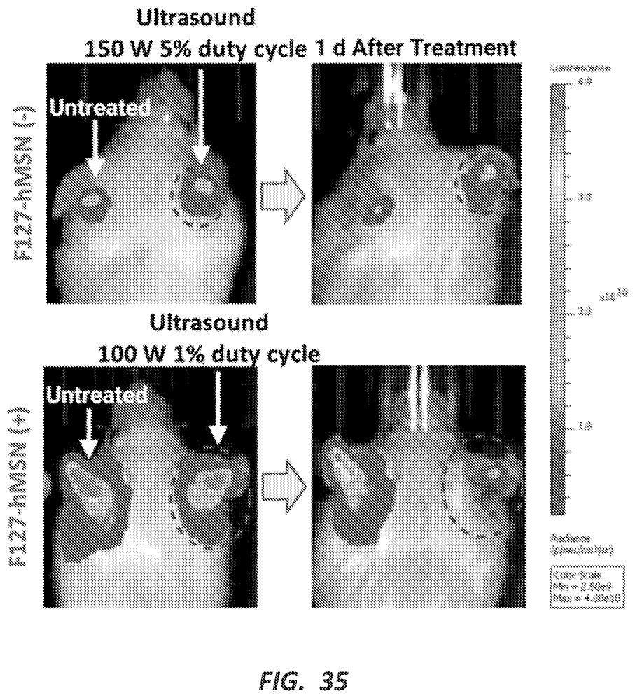

FIG. 35 shows luminescence images of mice bearing orthotopic A375 melanoma injected with or without F127-coated MSNs then treated with HIFU. In the absence of nanoparticles, the tumor size did not change significantly one day after treatment with HIFU (FIG. 35 upper panels). HIFU settings were 150 W of power input to the transducer and 100 μs long pulses at a repetition frequency of 500 Hz (duty cycle of 5%). In the presence of F127-hMSN, a significant reduction in the tumor size was observed one day after treatment using 7.5 fold lower acoustic intensities (FIG. 35 lower panels). HIFU settings were: 100 W of power input to the transducer, 20 μs long pulses at a repetition frequency of 500 Hz (duty cycle of 1%).

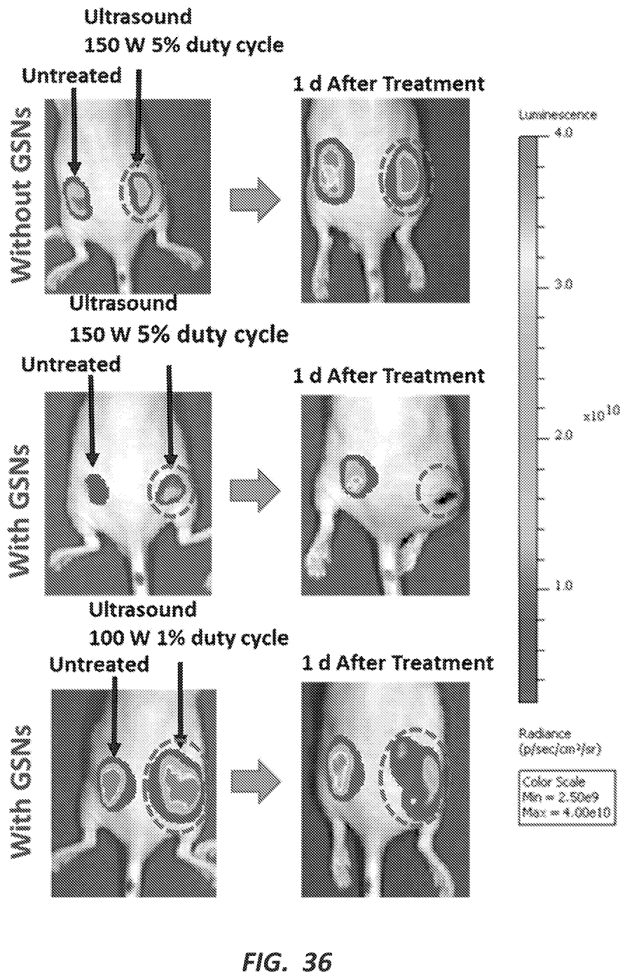

FIG. 36 shows luminescence images of the mice represented in FIG. 34. IVIS imaging before and after HIFU treatment showed no significant change in the tumor size when the tumors were treated without nanoparticle injection (FIG. 36 upper row). On the other hand, in the presence of MSA-hMSN, the tumor was almost completely ablated using the same HIFU settings (150 W, 5% duty cycle) (FIG. 36 center row). A significant reduction in the tumor size was still observed after reducing the acoustic intensity 5-fold (150 W, 1% duty cycle) (FIG. 36 lower row).

### EXAMPLES

### Example 1: Materials

Cetyltrimethylammonium chloride solution (25 wt. % in H₂O, CTAC), triethanolamine, potassium phosphate dibasic trihydrate (K₂HPO₄3H₂O), sodium bicarbonate (NaHCO₃), calcium chloride (CaCl₂)) and magnesium sulfate (MgSO₄) were purchased from Sigma-Aldrich. Hydrochloric Acid (36.5 to 38.0%, HCl), sodium chloride (NaCl), potassium chloride (KCl), tris(hydroxymethyl)aminomethane were purchased from Fisher Chemicals. Magnesium chloride (MgCl₂), tetraethyl orthosilicate (98%, TEOS), 1,1,1,3,3,3-hexamethyldisilazane (HMDS), ethanol (200 proof) were purchased from Alfa Aesar, Acros Organic, Gelest, and Decon Laboratories, respectively. Carbon Film 200 copper mesh was purchased from Electron Microscopy Sciences. RPMI-1640 cell culture medium, fetal bovine serum (FBS), Dulbecco's phosphate-buffered saline, and Calcein-AM/ethidium homodimer-1 live/dead assay were obtained from ThermoFisher Scientific. 3-(4,5-dimethylthiazol-2-yl)-2,5-diphenyltetrazolium bromide (MTS) cell proliferation assay was obtained from Promega. 4T1 mouse mammary gland cancer cells were purchased from the American Type Culture Collection.

### Example 2: MSN Synthesis

MSNs were prepared using previously published protocols with slight modifications. Briefly, in a round bottom flask 10 mL of CTAC (25% in water), 1.6 mL of freshly prepared triethanolamine solution (10% v/v, in ultrapure water), and 30 mL of ultrapure water were added and stirred at 600 rpm for 30 minutes at 80° C. Then, 3 mL of TEOS was added to the solution under stirring, and the solution was kept at the same conditions for 90 minutes before collecting the nanoparticles by centrifuging at 10000 rcf for 45 minutes. Then, the particles were washed twice with 35 mL of ethanol. Surfactant extraction was performed by stirring MSNs in an acidic ethanol solution (1.25% HCl) at 65° C. for at least 3 hours. This process was repeated three times to ensure complete surfactant removal. Finally, particles were dried in an oven at 65° C.

### Example 3: Synthesis of hMSNs

First, 50 mg of MSN was further dried at 150° C. for 4 h and dispersed in 8 mL of ethanol with 30 minutes of bath sonication. Then, 2 mL of HMDS was added, and the vial was heated to 50° C. under stirring and kept at this condition for 42 h. Particles were collected by centrifugation, washed twice with 30 mL of ethanol, and dried in an oven at 65° C. Finally, hMSN were suspended in ethanol to give a final concentration of 4 mg/mL.

### Example 4: Synthesis of F127-hMSNs

1 mL of hMSN dispersion in ethanol (4 mg/mL) was added to a 20 mL glass vial. First, the ethanol was evaporated by keeping the vial at 65° C. for at least 4 h. Then, the temperature was increased to 120° C. to remove any solvent trapped in the particles, and the vial was kept at this temperature for 1 h. After cooling down the vial to room temperature, 4 mL of PBS containing different amounts of F127 was added carefully to the top of the dried hMSN film. Finally, hMSN was suspended in PBS by 5 seconds of bath sonication, and the dispersions were stored at RT.

### Example 5: Nanoparticle Characterization

Dynamic light scattering (DLS) analysis of the nanoparticles dispersed in PBS was performed using a Zetasizer NANO (Malvern Pananaltical). Transmission electron microscopy (TEM) images were taken using FEI Tecnai microscope. TEM samples were prepared by drying the nanoparticle suspension in ethanol on carbon film 200 copper mesh TEM grids. The Raman spectra were recorded using XploRA PLUS confocal Raman microscopy system (HORIBA Scientific) equipped with a TE air-cooled CCD detector. The 638 nm excitation laser was derived from an air-cooled diode laser, and laser power was set to 3 mW on the sample. For Raman measurements, the F127-hMSN dispersion in PBS was washed several times with water to remove excess F127. The samples in water (MSN, F127-hMSN, F127) or ethanol (hMSN) were drop-casted on a CaF₂ glass slide and dried in an oven at 65° C. The laser was focused on the dried droplet surface using a 100×objective lens (NA=0.90). Raman photons were collected by the same objective lens into the spectrometer using an integration time of 10 seconds for all experiments.

### Example 6: Ultrasound Imaging of Nanoparticles

For ultrasound imaging, an IP-105 transducer (Sonic Concepts) operating at 2.5 MHz and different MIs (0.2-1.4) was submerged in a water tank along with the sample (2 mL), which is placed in the bulb of a plastic transfer pipette. B-mode images and 2 seconds long videos (at 100 Hz) were obtained via a Vantage 64 Research Ultrasound Imaging System (Verasonics) using a single plane wave transmit (1 cycle duration, ˜0.4 μs). ImageJ (NIH) was used to calculate the intensity of each frame of the recorded videos. Only the last 100 frames of the videos were analyzed to exclude the burst signal from the analysis. To calculate the average signal intensity, the minimum intensity value was subtracted from each frame, and then resulting frame intensities were averaged. Finally, the relative intensities were calculated by normalizing against the sample with the highest intensity. To estimate the peak negative pressure of ultrasound pulses at different driving voltages, a needle hydrophone (HNP-0400, ONDA) was used.

### Example 7: HIFU Insonation

For HIFU insonation, a single element HIFU transducer (Sonic Concepts H-102) with a center frequency of 1.1 MHz and equipped with a coupling cone (Sonic Concepts C-101) was used. The transducer was operated using a transducer power output system (TPO-102, Sonic Concepts). The HIFU transducer and cone were placed at the bottom of a water tank, and 2 mL of samples at different concentrations in the bulb of a plastic transfer pipette was placed to the focal zone of the transducer. The IP-105 transducer operating at 5 MHz and a MI of 0.26 was aligned to the sample to acquire videos during HIFU insonation. Then, HIFU was applied using the following parameters: center frequency of 1.1 MHz, 10 μs pulse duration, 10 Hz pulse repetition frequency, and at different power outputs between 5 and 50 W. To calculate the average intensities generated by the particles, the acquired videos were analyzed as described above. Here, all the recorded frames (200 total) were included to the analysis.

### Example 8: Testing Stability of Nanoparticles in Serum

In order to evaluate the stability of the ultrasound responsiveness of the F127-hMSN, 1 mL of FBS was mixed with 1 mL of F127-hMSN (200 μg/mL) in PBS. Particles were incubated overnight at 37° C. under slow rotation. Then, their ultrasound responsivity was evaluated as described above.

### Example 9: Ultrasound Imaging in Tissue-Mimicking Phantoms

To prepare tissue-mimicking agarose phantoms, first, agarose was dissolved in PBS at 90° C. under continuous stirring to give a final agarose concentration of 1.11% (w/w). When agarose dissolved completely, the solution was cooled down to 40° C. and 0.9 mL of this solution was mixed with 0.1 mL of F127-hMSN suspension in PBS (1 mg/mL) in the bulb of a plastic transfer pipette. Finally, the gels were allowed to solidify at RT and imaged.

### Example 10: Cell Culture

4T1 murine mammary carcinoma cells were cultured in RPMI-1640 supplemented with 10% FBS and 1% penicillin-streptomycin. Cells were incubated at 37° C. under 5% CO₂ atmosphere.

### Example 11: Flow Cytometry Evaluation of Effects of Cavitation on Cells

To evaluate the effects of cavitation on cells, 4T1 cells were collected by trypsinization and dispersed in serum-free RPMI-1640 to give a final cell number of 10⁶ cells/mL. Then, 0.1 mL of 1 mg/mL F127-hMSN solution in PBS was added to 0.9 mL of cell suspension for imaging experiments, and 0.05 mL of the same solution of F127-hMSN was added to 0.45 mL of cell suspension for HIFU experiments. Cells were insonated for 2 minutes for both modalities, as described above. Then, 4 μL of Ethidium homodimer (2 mM) and 2 μL of Calcein AM (50 μM in DMSO) solutions were added to 1 mL of samples and incubated at RT for 15-30 min. Finally, flow cytometry was performed using a BD FACS Symphony Analyzer. The scatter plot were divided into three regions, and the number of events in each region was recorded to calculate Count (%) values.

### Example 12: Testing Cell Viability in Response to Nanoparticles

For testing the viability of cells, the MTS assay used. First, 5×10³ 4T1 cells were added to each well of 96 well plates. Five wells per condition were prepared. The plate was incubated at 37° C. for 1 day. Then, the media were replaced with F127-hMSN containing RPMI-1640 (10% FBS) at different particle concentrations between 0 and 250 μg/mL. Cells with the particles were incubated for another day at 37° C., and then the media was replaced with 90 μL fresh media+10 μL MTS solution. The cells were then incubated at 37° C. for 2 h before recording the absorbance at 490 nm using a plate reader (TECAN Spark 20M).

### Example 13: Testing Nanoparticle Degradation

Degradation studies were performed in simulated body fluid (SBF), which was prepared according to a previous report with slight modifications. To prepare 1 L of SBF, 7.996 g of NaCl, 0.224 g of KCl, 0.228 g of K₂HPO₄₃H₂O were added to 700 mL of DI water and stirred at room temperature for 30 minutes. Then, 20 mL of HCl (2 M) was added to the solution among with 0.35 g of NaHCO₃, 0.1428 g of MgCl₂, 0.278 g of CaCl₂), 0.074 g of MgSO₄, 6.057 g of tris(hydroxymethyl)aminomethane. The pH of the solution was adjusted to 7.4, and DI water was added to make the final volume 1 L. The SBF solution was further stirred at room temperature for 2 hours. For the degradation studies, 250 μL of F127-hMSN in PBS (1 mg/mL) was diluted in 4.75 mL of SBF to give a final particle concentration of 50 μg/mL. Then, the particles were stirred at 37° C. for one week. Finally, the particles were collected by centrifugation at 10000 rcf for 45 minutes and washed twice with ethanol before TEM analysis.

### Example 14: Synthesis of More Biodegradable MSNs

Mesoporous silica nanoparticles (MSNs) with sizes around 50 nm were first synthesized as described in Example 2. A broad range of nanoparticles with different sizes and morphologies can be used. Then the surfaces of nanoparticles were hydrophobically modified using hexamethyldisilazane (HMDS). Other reactive silane monomers (such as chlorosilanes, methoxysilanes, ethoxysilanes, and disilazanes) can also be used to prepare hydrophobic nanoparticles. By tuning the hydrophobic monomer amount and the duration of the hydrophobic modification, biodegradable nanoparticles with high acoustic can be prepared. Specifically, the MSNs were incubated in 10% HMDS for 24 h at 50° C. to prepare hydrophobic nanoparticles. It can be possible to tune the biodegradation rate of ultrasound contrast agents using different hydrophobic monomers at different concentrations and reaction times. Decreasing the HMDS amount can further result in the formation of colloids with poor dispersibility and acoustic activity after stabilizing with proteins.

### Example 15: Preparation of Protein-Coated MSNs

For protein stabilization, first, the hydrophobic particles were dispersed in a suitable organic solvent (chloroform, ethanol, acetone etc.), and dried in a vial (glass, plastic, metal vials with different sizes and shapes) as described in Examples 3 and 4. The nanoparticles were modified using a reduced amount of hydrophobic monomer directly dispersed in water by brief bath sonication (around 5 seconds). When these nanoparticles are dispersed in buffer solutions such as PBS, they can aggregate due to the formation of salt bridges between nanoparticles. Thus, albumin (or other protein sources such as serum or plasma) was added to the PBS to prevent nanoparticle aggregation. In a typical procedure, 1 mL of hydrophobic nanoparticle dispersion (4 mg/mL in ethanol) was placed in a 20 mL glass vial and the ethanol was evaporated at 65° C. to form a thin film of nanoparticles. The dried films were further kept at 120° C. to remove any water or other solvents absorbed by the nanoparticles. Alternatively, particles can be kept under a vacuum to remove any adsorbed solvents. Then the nanoparticles were suspended in 2 mL of deionized or ultrapure water with a brief (around 5 seconds) bath sonication. Finally, particles were mixed with 2 mL of protein solution (1-50 mg/mL) in 2×PBS (20 mM, pH 7.4) and incubated at room temperature for 1-2 h to allow the formation of protein corona around nanoparticles. As a protein source, albumin (recombinant or purified) from different sources and human plasma were used. Potentially other protein sources such as fetal bovine serum or milk proteins may also be used.

### Example 16: Testing Ultrasound Responsiveness and Acoustic Activity of Coated MSNs

Hydrophobic MSNs coated with proteins from different sources were recorded under continuous ultrasound imaging for 2 min. Videos were recorded using an imaging transducer operating at 2.5 MHz at a mechanical index of 1.5, which is below the FDA limit (1.9) and available on conventional medical ultrasound instruments. Particles can be activated using a high intensity focused ultrasound (HIFU) transducer as described in Example 7.

### Example 17: Characterization of Protein-Coated Nanoparticles

Size distribution, degradation, and toxicity of nanoparticles were analyzed. Size distribution of protein-coated nanoparticles in PBS or uncoated particles in water was studied using dynamic light scattering (DLS) analysis as described in Example 5.

Degradation of protein-coated nanoparticles in simulated body fluid were tested as described in Example 13. Degradation of protein-coated nanoparticles were also tested in vivo. Cy7 dye labeled nanoparticles (100 μL, 10 mg/mL in saline) were injected intramuscularly to the legs of mice, and nanoparticle fluorescence was monitored for 54 days using an in vitro imaging system (IVIS). BSA coated hydrophobic MSNs, F127 polymer coated hydrophobic MSNs, and unmodified hydrophilic MSNs (Bare MSNs) were tested.

To evaluate toxicity of the nanoparticles, intravenous injection of the fluorescently labeled nanoparticles (200 μL, 10 mg/mL in saline) were performed into mice. Animals were sacrificed after 1 week, and the organ distribution of the fluorescent nanoparticles was detected using an IVIS.

### Example 18: Using Nanoparticles to Ablate Tumors

In one study, BSA coated hydrophobic MSNs were intratumorally injected (100-200 μL, 1 mg/mL in saline) in mice and HIFU treated immediately after injection. HIFU insonation was performed for 1 min using a HIFU transducer operating at 1.1 MHz. Two different pulse durations, 20 and 100 μs, were tested. Other HIFU conditions were the same for both treatments where input power was 150 W and PRF was 500 Hz. HCT-116 tumor volumes were measured.

In another experiment, mice bearing orthotopic A375 melanoma tumors on both ears were used. Luciferase expressing A375 cells were used to generate xenograft tumors to be able to monitor tumor size through bioluminescence imaging. F127 coated nanoparticles were intratumorally injected (100 μL, 1 mg/mL in saline), and the tumors were HIFU treated in a water tank filled with degassed water immediately after injection. Two mice were used in this experiment, where one mouse was HIFU insonated in the presence of F127-hMSN, and the other mouse was insonated in the absence of nanoparticles for 1 min. For both mice, the tumors on their right ears were HIFU treated, and the tumors on their left ears were left as untreated controls. HIFU settings were 150 W of power input to the transducer and 100 μs long pulses at a repetition frequency of 500 Hz (duty cycle of 5%) or 100 W of power input to the transducer, 20 μs long pulses at a repetition frequency of 500 Hz (duty cycle of 1%). To measure tumor sizes before and after HIFU treatment, luciferin solution was intravenously injected into the mice, and the luciferin bioluminescence was detected using an IVIS system.

In another study, mouse albumin coated hydrophobic MSNs (MSA-hMSN) were used to ablate the HCT-116 colon cancer xenografts in nude mice. Luciferase-expressing HCT-116 cells were used to grow two tumors on the backs of each mice. Nanoparticles were intratumorally injected (100 μL, 1 mg/mL in saline), and mice were treated with HIFU for 1 min with 150 W, 5% duty cycle or 150 W or 1% duty cycle. Only pulse duration was tuned to change the duty cycle while keeping pulse repetition frequency and power constant at 500 Hz and 150 W, respectively. Mice with tumors but not treated with nanoparticles were used as controls. IVIS imaging was performed before and after HIFU treatment.

These results indicate that the nanoparticles described herein can be potentially used to mechanically ablate solid tumors using relatively low acoustic intensities.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A hydrophobic nanoparticle for an ultrasound imaging contrast agent capable of being imaged by ultrasound equipment, the hydrophobic nanoparticle comprising:
a. a sub-100 nanometer nanoparticle core having an outer surface;
b. a silane layer coating the outer surface of the sub-100 nanometer nanoparticle core; and
c. a stabilization layer comprising stabilizing molecules, each stabilizing molecule in the stabilization layer having a binding portion bound to the silane layer and a non-binding portion free from the silane layer, the stabilizing molecules in the stabilization layer being spaced apart on the silane layer by distances configured to provide bubble nucleation sites that, in response to the ultrasound equipment delivering an acoustic intensity, initiate cavitation of echogenic micron-sized bubbles.

2. The hydrophobic nanoparticle of claim 1, wherein the acoustic intensity is delivered at a mechanical index of about 1.9 or less.

3. The hydrophobic nanoparticle of claim 2, wherein the acoustic intensity is delivered at a mechanical index of about 1.5 or less.

4. The hydrophobic nanoparticle of any of claims 1-3, wherein the sub-100 nanometer nanoparticle core is selected from the group consisting of silicon, silica, gold, silver, iron oxide, titanium dioxide, carbon, organosilica, a polymer, platinum, metal-organic framework, hydrogel, polydopamine, and cellulose.

5. The hydrophobic nanoparticle of any of claims 1-4, wherein the sub-100 nanometer nanoparticle core is mesoporous silica.

6. The hydrophobic nanoparticle of any of claims 1-5, wherein the sub-100 nanometer nanoparticle core is from about 30 nanometers to about 90 nanometers in diameter.

7. The hydrophobic nanoparticle of any of claims 1-6, wherein the sub-100 nanometer nanoparticle core is from about 30 nanometers to about 70 nanometers in diameter.

8. The hydrophobic nanoparticle of any of claims 1-7, wherein the sub-100 nanometer nanoparticle core is from about 40 nanometers to about 60 nanometers in diameter.

9. The hydrophobic nanoparticle of any of claims 1-8, wherein the sub-100 nanometer nanoparticle core is from about 45 nanometers to about 55 nanometers in diameter.

10. The hydrophobic nanoparticle of any of claims 1-9, wherein the sub-100 nanometer nanoparticle core is about 50 nanometers in diameter.

11. The hydrophobic nanoparticle of any of claims 1-10, wherein the silane layer comprises a silane selected from the group consisting of chlorosilanes, methoxysilanes, ethoxysilanes, and disilazanes.

12. The hydrophobic nanoparticle of any of claims 1-11, wherein the silane layer comprises hexamethyldisilazane.

13. The hydrophobic nanoparticle of any of claims 1-12, wherein the stabilizing molecules comprise one or more proteins selected from the group consisting of human serum albumin, bovine serum albumin, and egg albumin.

14. The hydrophobic nanoparticle of claim 13, wherein the stabilizing molecules comprise human serum albumin.

15. The hydrophobic nanoparticle of any of claims 1-7 and 11-13, wherein:
a. the sub-100 nanometer nanoparticle core is from about 30 nanometers to about 70 nanometers in diameter;
b. the silane layer comprises a silane selected from the group consisting of chlorosilanes, methoxysilanes, ethoxysilanes, and disilazanes; and
c. the stabilizing molecules comprise one or more proteins selected from the group consisting of human serum albumin, bovine serum albumin, and egg albumin.

16. The hydrophobic nanoparticle of any of claims 1-8, 11, 13 and 15, wherein:
a. the sub-100 nanometer nanoparticle core is from about 40 nanometers to about 60 nanometers in diameter;
b. the silane layer comprises a silane selected from the group consisting of chlorosilanes, methoxysilanes, ethoxysilanes, and disilazanes; and
c. the stabilizing molecules comprise one or more proteins selected from the group consisting of human serum albumin, bovine serum albumin, and egg albumin.

17. The hydrophobic nanoparticle of any of claims 1-9, 11, 13 and 16, wherein:
a. the sub-100 nanometer nanoparticle core is from about 40 nanometers to about 55 nanometers in diameter;
b. the silane layer comprises a silane selected from the group of chlorosilanes, methoxysilanes, ethoxysilanes, and disilazanes; and
c. the stabilizing molecules comprise one or more proteins selected from the group consisting of human serum albumin, bovine serum albumin, and egg albumin.

18. The hydrophobic nanoparticle of any of claims 1-8, 11, and 13-16, wherein:
a. the sub-100 nanometer nanoparticle core is from about 40 nanometers to about 60 nanometers in diameter;
b. the silane layer comprises a silane selected from the group consisting of chlorosilanes, methoxysilanes, ethoxysilanes, and disilazanes; and
c. the stabilizing molecules comprise human serum albumin.

19. The hydrophobic nanoparticle of any of claims 1-8, 12, 13, and 16, wherein:
a. the sub-100 nanometer nanoparticle core is a mesoporous silica nanoparticle core of from about 40 nanometers to about 60 nanometers in diameter;
b. the silane layer comprises hexamethyldisilazane; and
c. the stabilizing molecules comprise one or more proteins selected from human serum albumin and bovine serum albumin.

20. The hydrophobic nanoparticle of any of claims 1-12, wherein the stabilizing molecules comprise one or more amphiphilic molecule chains selected from the group consisting of poloxamers, poly(D,L-lactide-co-glycolide), and phospholipids.

21. The hydrophobic nanoparticle of claim 20, wherein the amphiphilic molecule chains comprise a poloxamer selected from the group consisting of poloxamer 188, poloxamer 237, poloxamer 338, and poloxamer 407.

22. The hydrophobic nanoparticle of claim 21, wherein the amphiphilic molecule chains comprise poloxamer 407.

23. The hydrophobic nanoparticle of any of claims 1-7, 11 and 22, wherein:
a. the sub-100 nanometer nanoparticle core is from about 30 nanometers to about 70 nanometers in diameter;
b. the silane layer comprises a silane selected from the group consisting of chlorosilanes, methoxysilanes, ethoxysilanes, and disilazanes; and
c. the stabilizing molecules comprise poloxamer 407.

24. The hydrophobic nanoparticle of any of claims 1-23, wherein said hydrophobic nanoparticle substantially completely biodegrades within a period of less than one month after introduction into a body of a subject.

25. A method of preparing a stabilized hydrophobic nanoparticle for an ultrasound imaging contrast agent capable of being imaged by ultrasound equipment, the method comprising the steps of:
a. treating the outer surface of a sub-100 nanometer nanoparticle core with one or more silane monomers selected from the group of chlorosilanes, methoxysilanes, ethoxysilanes, and disilazanes to create a hydrophobic nanoparticle;
b. dispersing the hydrophobic nanoparticle in an acceptable organic solvent to create a composition with a final particle concentration of from about 1 mg/mL to about 6 mg/mL;
c. removing the acceptable organic solvent from the composition to provide a dried nanoparticle film; and
d. treating the dried nanoparticle film with a stabilizing molecule solution to create a stabilization layer on the sub-100 nanometer nanoparticle.

26. The method of claim 25, wherein the one or more silane monomers comprises hexamethyldisilazane.

27. The method of any of claims 25 and 26, wherein the acceptable organic solvent comprises ethanol.

28. The method of any of claims 25-27, wherein the stabilizing molecule solution is a protein solution of from about 0.5 mg/mL to about 100 mg/mL of protein.

29. The method of claim 28, wherein the protein solution comprises one or more proteins selected from the group of human serum albumin, bovine serum albumin, and egg albumin.

30. The method of any of claims 28 and 29, wherein the protein solution comprises one or more proteins selected from the group of human serum albumin, bovine serum albumin, and egg albumin.

31. The method of any of claims 28-30, wherein the protein solution comprises protein at a concentration of from about 0.5 mg/mL to about 75 mg/mL of protein.

32. The method of any of claims 28-31, wherein the protein solution comprises protein at a concentration of from about 0.5 mg/mL to about 60 mg/mL of protein.

33. The method of any of claims 28-32, wherein the protein solution comprises protein at a concentration of from about 1 mg/mL to about 50 mg/mL of protein.

34. The method of any of claims 28-33, wherein treating the dried nanoparticle film comprises:
a. adding an amount of water to the dried nanoparticle film;
b. sonicating the water and the dried nanoparticle film to produce a suspension of hydrophobic nanoparticles; and
c. adding the protein solution to the suspension.

35. The method of any of claims 25-27, wherein the stabilizing molecule solution comprises one or more amphiphilic molecule chains selected from the group consisting of poloxamers, poly(D, L-lactide-co-glycolide), and phospholipids.

36. The method of claim 35, wherein the amphiphilic molecule chains comprise a poloxamer selected from the group consisting of poloxamer 188, poloxamer 237, poloxamer 338, and poloxamer 407.

37. The method of claim 36, wherein the amphiphilic molecule chains comprise poloxamer 407.

38. The method of any of claims 35-37, wherein the stabilizing molecule solution comprises amphiphilic molecule chains at a concentration of about 0.25 mg/mL to about 2 mg/mL.

39. The method of any of claims 35-38, wherein the stabilizing molecule solution comprises amphiphilic molecule chains at a concentration of about 0.25 mg/mL to about 0.5 mg/mL.

40. The method of any of claims 35-39, wherein the weight ratio of the amphiphilic molecule chains to the dried nanoparticle film is about 1:2 to about 1:4.

41. The method of any of claims 25-40, wherein the sub-100 nanometer nanoparticle comprises mesoporous silica.

42. A method of using the hydrophobic nanoparticle of any of claims 1-24 as the ultrasound imaging contrast agent, the method comprising:
administering the hydrophobic nanoparticle to a subject at a concentration in a range of about 1 μg/mL to about 5 mg/mL.

43. The method of claim 42, wherein the concentration is about 2.5 μg/mL to about 100 μg/mL.

44. A method of using the hydrophobic nanoparticle of any of claims 1-24 in high intensity focused ultrasound (HIFU) ablation therapy, the method comprising:
a. delivering the hydrophobic nanoparticle to a target tissue at a concentration of about 0.05 mg/mL to about 10 mg/mL;
b. insonating the target tissue with HIFU to reduce a volume of the target tissue.
